SEQUENCE LISTING (38 sequences)

SEQ ID NO: 1 - DNA, 20 bp, Artificial Sequence
  oligonucleotide
gtctgggcggtgctacaact

SEQ ID NO: 2 - DNA, 20 bp, Artificial Sequence
  oligonucleotide
tgcctgtacaagctccacaa

SEQ ID NO: 3 - DNA, 20 bp, Artificial Sequence
  oligonucleotide
gagaggaaatccgtccattg

SEQ ID NO: 4 - DNA, 20 bp, Artificial Sequence
  oligonucleotide
tgtgaacaacctaaactgat

SEQ ID NO: 5 - DNA, 20 bp, Artificial Sequence
  oligonucleotide
tgcctgtgcaggcagctcaa

SEQ ID NO: 6 - DNA, 20 bp, Artificial Sequence
  oligonucleotide
agcttccacaatgcctgtgc

SEQ ID NO: 7 - DNA, 20 bp, Artificial Sequence
  oligonucleotide
ctccaccgtgatgtacaccg

SEQ ID NO: 8 - DNA, 20 bp, Artificial Sequence
  oligonucleotide
ctcctcggtgtacatcacgg

SEQ ID NO: 9 - DNA, 20 bp, Artificial Sequence
  oligonucleotide
gtgactgacatcaactccaa

SEQ ID NO: 10 - DNA, 20 bp, Artificial Sequence
  oligonucleotide
ggagttgatgtcagtcactt

SEQ ID NO: 11 - DNA, 20 bp, Artificial Sequence
  oligonucleotide
ctcgatggtgaatgacagtg

SEQ ID NO: 12 - DNA, 20 bp, Artificial Sequence
  oligonucleotide
ggtgaatgacagtgcggttg

SEQ ID NO: 13 - DNA, 20 bp, Artificial Sequence
  oligonucleotide
ccatcgagtgccaaatgaag

SEQ ID NO: 14 - DNA, 20 bp, Artificial Sequence
  oligonucleotide
gcttctgctgccggttaacg

SEQ ID NO: 15 - DNA, 20 bp, Artificial Sequence
  oligonucleotide
ttgaactcagcttctgctgc

SEQ ID NO: 16 - DNA, 20 bp, Artificial Sequence
  oligonucleotide
gcagaagctgagttcaacct

SEQ ID NO: 17 - DNA, 66 bp, Artificial Sequence
  oligonucleotide, “N” represents any nucleotide
  misc_feature (37)..(37): n is a, c, g, or t
  misc_feature (43)..(43): n is a, c, g, or t
  misc_feature (56)..(56): n is a, c, g, or t
  misc_feature (59)..(60): n is a, c, g, or t
  misc_feature (66)..(66): n is a, c, g, or t
ataatgaatttgatgagaatgtgcacaaacccatcangttaangttgttcacacantgnntgcaan

SEQ ID NO: 18 - DNA, 66 bp, Artificial Sequence
  oligonucleotide, “N” represents any nucleotide
ataatgaatttgatgagaatgtgcacaaacccatcagtttaggttgttcacacactgtttgcaaga

SEQ ID NO: 19 - DNA, 75 bp, Artificial Sequence
  oligonucleotide, “N” represents any nucleotide
  misc_feature (26)..(26): n is a, c, g, or t
tgatgagaatgtgcacaaacccatcnagtttaggttgttcacacactgtttgcaagacctgcttgaataaacttc

SEQ ID NO: 20 - DNA, 49 bp, Artificial Sequence
  oligonucleotide, “N” represents any nucleotide
tgatgagaatgtgcaacactgtttgcaagacctgcttgaataaacttca

SEQ ID NO: 21 - DNA, 73 bp, Artificial Sequence
  oligonucleotide, “N” represents any nucleotide
tgatgagaatgtgcacaaacccatctttaggttgttcacacactgtttgcaagacctgcttgaataaacttca

SEQ ID NO: 22 - DNA, 74 bp, Artificial Sequence
  oligonucleotide, “N” represents any nucleotide
tgatgagaatgtgcacaaacccatcgtttaggttgttcacacactgtttgcaagacctgcttgaataaacttca

SEQ ID NO: 23 - DNA, 70 bp, Artificial Sequence
  oligonucleotide, “N” represents any nucleotide
tgatgagaatgtgcacaaacccatcaggttgttcacacactgtttgcaagacctgcttgaataaacttca

SEQ ID NO: 24 - DNA, 75 bp, Artificial Sequence
  oligonucleotide, “N” represents any nucleotide
tgatgagaatgtgcacaaacccatcagtttaggttgttcacacactgtttgcaagacctgcttgaataaacttca

SEQ ID NO: 25 - DNA, 56 bp, Artificial Sequence
  oligonucleotide, “N” represents any nucleotide
tgatgagaatgtgcacaaacccacactgtttgcaagacctgcttgaataaacttca

SEQ ID NO: 26 - DNA, 72 bp, Artificial Sequence
  oligonucleotide, “N” represents any nucleotide
tgatgagaatgtgcacaaacccatcttaggttgttcacacactgtttgcaagacctgcttgaataaacttca

SEQ ID NO: 27 - DNA, 65 bp, Artificial Sequence
  oligonucleotide, “N” represents any nucleotide
ttgtaactccagaagaatgcctgtacaagctccaacaagggacggatttcccctccggcccattt

SEQ ID NO: 28 - DNA, 65 bp, Artificial Sequence
  oligonucleotide, “N” represents any nucleotide
ttgtaactccagaagaatgcctgtacaagctccacaatggacggatttcctctcctgcccaattt

SEQ ID NO: 29 - DNA, 66 bp, Artificial Sequence
  oligonucleotide, “N” represents any nucleotide
ataatgaatttgatgagaatgtgcacaaacccatcaagtttaggttgttcacacactgtttgcaaa

SEQ ID NO: 30 - DNA, 66 bp, Artificial Sequence; identical to SEQ ID NO: 18
  oligonucleotide, “N” represents any nucleotide

SEQ ID NO: 31 - DNA, 67 bp, Artificial Sequence
  oligonucleotide, “N” represents any nucleotide
ggccatgcccatcatgggctcctcggttgtacatcacggtggagctggccattgctgtgctggccat

SEQ ID NO: 32 - DNA, 66 bp, Artificial Sequence
  oligonucleotide, “N” represents any nucleotide
ggccatgcccatcatgggctcctcggtgtacatcacggtggagctggccattgctgtgctggccat

SEQ ID NO: 33 - DNA, 75 bp, Artificial Sequence
  oligonucleotide, “N” represents any nucleotide
  misc_feature (26)..(26): n is a, c, g, or t
atcatgggctcctcggtgtacatcancggtggagctggccattgctgtgctggccatcctgggcaatgtgctggt

SEQ ID NO: 34 - DNA, 63 bp, Artificial Sequence
  oligonucleotide, “N” represents any nucleotide
atcatgggctcctcggtggagctggccattgctgtgctggccatcctgggcaatgtgctggtg

SEQ ID NO: 35 - DNA, 75 bp, Artificial Sequence
  oligonucleotide, “N” represents any nucleotide
atcatgggctcctcggtgtacatcacggtggagctggccattgctgtgctggccatcctgggcaatgtgctggtg

SEQ ID NO: 36 - DNA, 57 bp, Artificial Sequence
  oligonucleotide, “N” represents any nucleotide
atcatgggctcctcggttggccattgctgtgctggccatcctgggcaatgtgctggt

SEQ ID NO: 37 - DNA, 64 bp, Artificial Sequence
  oligonucleotide, “N” represents any nucleotide
atcatgggctcctcggttggagctggccattgctgtgctggccatcctgggcaatgtgctggtg

SEQ ID NO: 38 - DNA, 66 bp, Artificial Sequence
  oligonucleotide, “N” represents any nucleotide
atcatgggctcctcggtggtggagctggccattgctgtgctggccatcctgggcaatgtgctggtg

(19) United States
(12) Patent Application Publication
(10) Pub. No.: US 2023/0009232 A1
(43) Pub. Date: Jan. 12, 2023

(54) METHOD FOR PROVIDING IMMUNE CELLS WITH ENHANCED FUNCTION

(71) Applicant: CARTHERICS PTY. LTD., Carlton, Victoria (AU)

(72) Inventors: Runzhe SHU, Carlton, Victoria (AU); Alan Osborne TROUNSON, Carlton, Victoria (AU); Richard BOYD, Carlton, Victoria (AU); Ian NISBET, Carlton, Victoria (AU); Nicholas BOYD, Carlton, Victoria (AU); Vera EVTIMOV, Carlton, Victoria (AU)

(73) Assignee: CARTHERICS PTY. LTD., Carlton, Victoria (AU)

(21) Appl. No.: 17/778,064

(22) PCT Filed: Nov. 18, 2020
(86) PCT No.: PCT/AU2020/051243

§ 371 Date: May 19, 2022

Related U.S. Application Data
(60) Provisional application No. 62/938,022, filed on Nov. 20, 2019.

(51) Int. Cl. C12N 15/90; C12N 5/0783; C12N 15/113; C12N 5/074; C12N 9/22; C12N 15/11; A61K 35/17; A61P 35/00

(52) U.S. Cl. CPC C12N 15/907; C12N 5/0636; C12N 15/113; C12N 5/0696; C12N 9/22; C12N 15/11; A61K 35/17; C12N 5/0646; A61P 35/00; C12N 2310/20; C12N 2506/45; C12N 2501/65

(57) ABSTRACT

This disclosure relates to methods for producing immune cells with enhanced function. More specifically, disclosed herein is a method for enhancing the function of an immune cell comprising modifying an immune cell to inhibit the function of at least one gene selected from the group consisting of RC3H1, RC3H2, A2AR, FAS, TGFBR1, and TGFBR2. Also disclosed herein is a method comprising modifying a stem or progenitor cell capable of differentiating into an immune cell to inhibit the function of at least one gene selected from the group consisting of RC3H1, RC3H2, A2AR, FAS, TGFBR1, and TGFBR2. Also disclosed herein are immune cells or stem cells made by the present methods, as well as the use of immune cells in therapeutic treatment.

### CROSS REFERENCE TO RELATED APPLICATION

This application claims the benefit of priority from U.S. Provisional Application No. 62/938,022, filed Nov. 20, 2019, the contents of which are incorporated herein in their entirety.

### FIELD OF THE DISCLOSURE

This disclosure relates to methods for producing immune cells with enhanced function. More specifically, disclosed herein is a method for enhancing the function of an immune cell comprising modifying an immune cell to inhibit the function of at least one gene selected from the group consisting of RC3H1, RC3H2, A2AR, FAS, TGFBR1, and TGFBR2. Also disclosed herein is a method comprising modifying a stem or progenitor cell capable of differentiating into an immune cell to inhibit the function of at least one gene selected from the group consisting of RC3H1, RC3H2, A2AR, FAS, TGFBR1, and TGFBR2. Also disclosed herein are immune cells or stem cells made by the present methods, as well as the use of immune cells in therapeutic treatment.

### INCORPORATION BY REFERENCE OF SEQUENCE LISTING

The Sequence Listing in the ASCII text file, named as 37830WO_ND201903_SequenceListing.txt of 10 KB, created on Nov. 3, 2020, is incorporated herein by reference.

### BACKGROUND ART

T cells expressing chimeric antigen receptors (CAR-T cells) have been shown to be very effective in killing tumor cells in diseases such as acute lymphocytic leukemia (ALL) and non-Hodgkin's lymphoma (NHL). Approved products targeting the B cell antigen CD19 are produced by introducing a CAR gene construct into patient-derived (“autologous”) T cells (Kershaw et al., Gene-engineered T cells for cancer therapy, Nat Rev Cancer, 2013, 13(8): 52541). Additional autologous products are in development targeting other blood cell markers such as B cell maturation antigen (BCMA) for other hematological malignancies, such as multiple myeloma (Sadelain et al., Therapeutic T cell engineering, Nature, 2017, 545(7655): 423-431).

While the clinical results with CAR-T cells in blood-based cancers have been impressive, similar results have not been forthcoming in the treatment of solid tumors. There are multiple reasons for the relative lack of efficacy in solid tumors, including restricted access to the tumor site, the immunosuppressive nature of the tumor microenvironment and the lack of solid tumor-specific target antigens. In addition, lack of persistence and “exhaustion” of the administered CAR-T cells is a consistently observed limitation (Newick et al., CAR T Cell Therapy for Solid Tumors, Annu Rev Med, 2017, 68: 139-152).

Inhibitory receptors like CTLA-4, PD-1, or LAG-3 can attenuate the activation of CAR-T cells and accelerate T cell exhaustion. An improved anti-tumor activity of T cells was expected after PD-1 was disrupted by genome editing (Liu et al., CRISPR-Cas9-mediated multiplex gene editing in CAR-T cells, Cell Res, 2017, 27(1): 154-157). However, ablation of PD-1 on T cells may also increase the susceptibility to exhaustion, reduce the longevity and fail to improve anti-tumor effect (Odorizzi et al., Genetic absence of PD-1 promotes accumulation of terminally diferentiated exhausted CD8+ T cells, J Exp Med, 2015, 212(7): 1125-37). For these reasons, whether gene editing in T cells will enhance anti-tumor activity or not needs to be evaluated case-by-case.

CRISPR/Cas9 is an important component of the bacterial immune system that allows bacteria to remember and destroy bacteriophages. In mammalian cells, CRISPR/Cas9 could be applied for gene editing like other gene editing technologies, such as TALEN and ZFN. CRISPR system contains two major components, the Cas9 nuclease and guide RNA. Specifically, designed guide RNAs form a complex with Cas9 nuclease guide Cas9-gRNA ribonucleoprotein (RNP) complex to a user defined cut site in the human genome. The RNP cutting results in a double strand DNA break in the genome, and the double strand DNA break is repaired by an error-prone process called Non-Homologous End Joining (NHEJ). In NHEJ pathway, nucleotide deletions or insertions (“indels”) result in gene disruption or knock-out (Addgene, CRISPR 101: A Desktop Resource (2nd Edition), 2017). The on-target efficiency and off-target effects of a guide RNA determine the specificity and safety of a CRISPR/Cas9 gene targeting application. As a result, a specially designed guide RNA plays a crucial role in the success of the gene disruption.

A recent study performed a genome-wide loss-of-function screen of immune regulators using CRISPR, and discovered that ablation of negative regulators like TCE2, SOCS1, RASA and CBLB, significantly increased T cell cytotoxicity in vitro (Shifrut et al., Genome-wide CRISPR Screens in Primary Human T Cells Reveal Key Regulators of Immune Function. Cell, 2018, 175(7): 1958-1971, e15). However, short-term in vitro cytotoxicity provides limited guidance on the effect of gene inhibition or deletion on in vivo function or longevity. The potential effects that the inhibition of a gene may have on the function of an immune cell (including T cell, NK cell, NKT cell, etc) including its activity or longevity, need to be assessed more broadly in vitro and in vivo.

While as enhanced immune cells are a potential weapon against cancer, there are challenges to numerically generate, expand and characterise immune cell products. Immune cells can be generated from pluripotent stem cells (PSCs). Pluripotent stem cell technology is therefore a very promising technology as, theoretically, pluripotent stem cells provide an unlimited, renewable source of cells. The ability to directly generate an effectively limitless supply of immune cells from stem cells (e.g. induced pluripotent stem cells (iPSCs)), with enhanced capabilities, also including a broad set of target recognition systems (TCR/CAR/cytotoxic receptors) capable of responding to multiple pathogens and also cancer, represents a major commercial opportunity. Therefore, it is also relevant to understand the impact of the inhibition of a particular gene of interest on iPSC viability, self-renewal, proliferation ability and capacity to differentiate into an immune cell.

### SUMMARY OF THE DISCLOSURE

It has been demonstrated herein that the inhibition of several genes enhanced the persistence and anti-tumor activity of cytotoxic cells in vivo.

In one aspect, provided herein is a method for enhancing the function of an immune cell. The method comprises modifying the immune cell to inhibit the function of at least one gene (i.e., one or more genes) selected from the group consisting of RC3H1, RC3H2, A2AR, FAS, TGFBR1 and TGFBR2.

In another aspect, provided herein is a method of modifying a stem cell capable of differentiating to an immune cell. The method comprises modifying the stem cell to inhibit the function of at least one gene selected from the group consisting of RC3H1, RC3H2, A2AR, FAS, TGFBR1 and TGFBR2. In some embodiments, a modified stem cell is further differentiated into an immune cell, wherein the function of said at least one gene is inhibited in the immune cell.

In some embodiments, inhibition of the function of a gene is achieved by reducing the level or function of mRNA, optionally through a small interfering RNA (siRNA), a short hairpin RNA (shRNA), a microRNA (miRNA), or an anti-sense nucleic acid.

In some embodiments, inhibition of the function of a gene is achieved by reducing the level or activity of the protein encoded by the gene, optionally through the use of an antibody or a small molecule.

In some embodiments, inhibition of the function of a gene is achieved by a gene editing system. In some embodiments, the gene editing system is selected from the group consisting of CRISPR/Cas, TALEN and ZFN. In some embodiments, the gene editing system is a CRISPR/Cas system which comprises a guide RNA-nuclease complex. In some embodiments, the guide RNA targets a nucleotide sequence selected from the group consisting of SEQ ID NO: 2 to SEQ ID NO: 16. In some embodiments, the CRISPR/Cas system utilizes a guide RNA dependent nuclease selected from the group consisting of Cpf1, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the immune cell is selected from a T cell (including cells such as an NKT cell), or an NK cell.

In some embodiments, a modified cell produced by the method disclosed herein, such as a modified immune cell or a modified stem cell, further comprises a nucleic acid encoding a chimeric antigen receptor (CAR).

In some embodiments, a modified immune cell produced by the method disclosed herein recognizes one or more target antigens. In some embodiments, the target antigens are selected from the group consisting of TAG-72, CD19, CD20, CD24, CD30, CD47, folate receptor alpha (FRα), and BCMA.

In a further aspect, provided herein is an immune cell produced by a method disclosed herein.

In another aspect, provided herein is a modified stem cell produced by a method disclosed herein.

In one aspect, provided herein is a modified immune cell, wherein the function of at least one gene is inhibited in the modified immune cell relative to an unmodified immune cell, wherein the at least one (i.e., one or more) gene is selected from the group consisting of RC3H1, RC3H2, A2AR, FAS, TGFBR1 and TGFBR2. In some embodiments, the RC3H1 gene is inhibited in a modified immune cell. In some embodiments, the RC3H2 gene is inhibited in a modified immune cell. In some embodiments, the A2AR gene is inhibited in a modified immune cell. In some embodiments, the FAS gene is inhibited in a modified immune cell. In some embodiments, the TGFBR1 gene is inhibited in a modified immune cell. In some embodiments, the TGFBR2 gene is inhibited in a modified immune cell. In some embodiments, multiple genes selected from RC3H1, RC3H2, A2AR, FAS, TGFBR1 and TGFBR2 are inhibited.

In some embodiments, the inhibition of the function of a gene in a modified immune cell, results from a reduction in the level or function of the mRNA transcribed from the gene, or the level or activity of the protein encoded by the gene.

In some embodiments, the inhibition of the function of a gene results from a modification in the nucleic acid sequence of the gene.

In some embodiments, the modified immune cell is selected from a T cell (including cells such as an NKT cell), or an NK cell.

In some embodiments, the modified immune cell expresses a chimeric antigen receptor (CAR).

In some embodiments, the modified immune cell recognizes one or more target antigens. In some embodiments, the target antigen is selected from the group consisting of TAG-72, CD19, CD20, CD24, CD30, CD47, folate receptor alpha (FRα) and BCMA.

In another aspect, provided herein is a modified stem cell, capable of differentiating to an immune cell, comprising a modification in the nucleic acid sequence of at least one gene, wherein the modification inhibits the function of the at least one gene and wherein the at least one gene is selected from the group consisting of RC3H1, RC3H2, A2AR, FAS, TGFBR1 and TGFBR2.

In some embodiments, the RC3H1 gene is inhibited in a modified stem cell. In some embodiments, the RC3H2 gene is inhibited in a modified stem cell. In some embodiments, the A2AR gene is inhibited in a modified stem cell. In some embodiments, the FAS gene is inhibited in a modified stem cell. In some embodiments, the TGFBR1 gene is inhibited in a modified stem cell. In some embodiments, the TGFBR2 gene is inhibited in a modified stem cell. In some embodiments, multiple genes selected from RC3H1, RC3H2, A2AR, FAS, TGFBR1 and TGFBR2 are inhibited.

In some embodiments, the modified stem cell is an induced pluripotent stem cell.

In some embodiments, a modified stem cell comprises a nucleic acid encoding a chimeric antigen receptor (CAR).

In a further aspect, provided herein is a composition for enhancing the function of an immune cell, comprising a guide RNA-nuclease complex capable of editing the sequence of a target gene, wherein the guide RNA targets a nucleotide sequence selected from the group consisting of SEQ ID NO: 2 to SEQ ID NO: 16.

In some embodiments, the nuclease comprises at least one protein selected from the group consisting of Cpf1, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In another aspect, provided is a method of treating a condition in a subject, comprising administering to the subject a modified immune cell disclosed herein. In some embodiments, the condition is a cancer, an infection, an autoimmune disorder, fibrosis of an organ, or endometriosis.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1B. An exemplified strategy of evaluating the anti-tumor activity of modified immune cells. (A) Schematic representation for the strategy implemented for the evaluation of CAR-T cells comprising a CRISPR knock-out of an immune regulatory gene, showing a representative timeline of the lentiviral CAR transduction, gene targeting and functional analyses in primary T cells used in the examples. (B) Representative timeline of the generation of modified NK-92 cells where CRISPR knock-out of an immune regulatory gene was followed by lentiviral CAR transduction and functional analysis in NK-92 cells.

FIGS. 2A-2B. Lentiviral transduction of human primary T cells to generate TAG-72 CAR-T cells. (A) Schematic diagram of the TAG-72-specific CAR construct used in this study. (B) Transduction efficiency of CAR in primary human T cells. Expression was examined 10 days following transduction with the lentiviral vector. Values embedded within each dotplot represent the frequency of CAR+ events as a percent of viable, single cells. (Representative data of the T cells from one donor are shown).

FIG. 3. Growth curve of the TAG-72 CAR-T cells after CRISPR/Cas9 RNP transfection (representative data of the T cells from one donor are shown). NT: non-transduced T cells; TAG-72 CAR: T cells transduced with a TAG-72 CAR; TAG-72 CAR/PD-1 KO T: T cells transduced with a TAG-72 CAR and CRISPR/Cas9 RNP targeting PD-1; TAG-72 CAR/A2AR KO T: T cells transduced with a TAG-72 CAR and CRISPR/Cas9 RNP targeting A2AR; TAG-72 CAR/FAS KO T: T cells transduced with a TAG-72 CAR and CRISPR/Cas9 RNP targeting FAS; TAG-72 CAR/RC3H1 KO T: T cells transduced with a TAG-72 CAR and CRISPR/Cas9 RNP targeting RC3H1; TAG-72 CAR/RC3H2 KO T: T cells transduced with a TAG-72 CAR and CRISPR/Cas9 RNP targeting RC3H2; TAG-72 CAR/TGFBR1 KO T: T cells transduced with a TAG-72 CAR and CRISPR/Cas9 RNP introducing dominant negative mutation into TGFBR1; TAG-72 CAR/TGFBR2 KO T: T cells transduced with a TAG-72 CAR and CRISPR/Cas9 RNP introducing dominant negative mutation into TGFBR2.

FIGS. 4A-4D. Transfection of guide RNAs formed RNP introduces insertions and deletions (indels) into the open reading frame of the specific genes in CAR-T cells. Frequency of indels was assessed by Inference of CRISPR Edits (ICE) assay. (A) Sanger sequencing trace from the RC3H2 gRNA transfected CAR-T cells (“edited sample”) shows a heterogeneous mix of bases downstream of the cut site in contrast to the non-transfected CAR-T cells (“control sample”) (SEQ ID NO: 17 sets forth 281 to 346 bp from the edited sample; SEQ ID NO: 18 sets forth 281 to 346 bp from the control sample). The black underlined region of the control sample represents the guide sequence and the horizontal red dotted underlined region is the associated PAM (Protospacer Adjacent Motif) site. The vertical black dotted line on both traces represents the cut site. (B) Relative percentage of the contribution of each edited sequence (normalized) in the genomic DNA from RC3H2 RNP transfected CAR-T cells. The sequences from top to bottom are set forth in SEQ ID NO: 19, 20, 21, 22, 23, 24, 25 and 26, respectively. (C) Distribution of the indel sizes in the entire edited population of RC3H2 RNP transfected CAR-T cells. Out-of-frame indel percentage is the proportion of indels that indicate a frameshift or are more than 21 bp in length. R² value computed by Pearson correlation coefficient indicates the confidence of the indel percentage. (D) Summary of the ICE assay result of the RNP transfected CAR-T cells. RNP complexes were formed by the representative guide RNAs used in this study (PD-1, SEQ ID NO: 1; RC3H1, SEQ ID NO: 2; RC3H2, SEQ ID NO: 4; A2AR, SEQ ID NO: 7; FAS, SEQ ID NO: 9; TGBFBR1, SEQ ID NO: 11; TGFBR2, SEQ ID NO: 14; representative data of the T cells from one donor are shown.

FIGS. 5A-5H. Gene knock out TAG-72 CAR-T cells mediate potent cell killing of TAG-72hi expressing target cells (OVCAR-3 cell line) (FIGS. 5A, 5C, 5E and 5G), but not TAG-72-neg/low cancer target cells (MES-OV cell line) (FIGS. 5B, 5D, 5F and 5H). Target cells were allowed to adhere to plates overnight before addition of CAR-T cells at an effector to target ratio of 1:1. Non-transduced T cells (NT) were included in the killing assay as controls. Cell impedance (mean±SD, represented as Normalised Cell Index (NCI)) was monitored over 20 h. Target cell proliferation under normal growth conditions (“Target cells only”) was also monitored throughout. (Representative data of the T cells from one donor performed in technical triplicate are shown). CAR-T (FIGS. 5A-5H): TAG-72 CAR-T cells; PD-1 (FIGS. 5A-5B): PD-1 knock-out TAG-72 CAR-T cells; RC3H1 (FIGS. 5C and 5D): RC3H1 knock-out TAG-72 CAR-T cells; RC3H2 (FIGS. 5C and 5D): RC3H2 knock-out TAG-72 CAR-T cells; A2AR (FIGS. 5E and 5F): A2AR knock-out TAG-72 CAR-T cells; FAS (FIGS. 5E and 5F): FAS knock-out TAG-72 CAR-T cells; TGFBR1 (FIGS. 5G and 5H): TGFBR1 dominant negative TAG-72 CAR-T cells; TGFBR2 (FIGS. 5G and 5H): TGFBR2 dominant negative TAG-72 CAR-T cells.

FIG. 6. Tumor growth curve of OVCAR-3 ovarian tumor in NOD scid gamma (NSG) mice xenograft models. Four NSG mice per group were subcutaneously administered 1×10⁷ OVCAR-3 tumor cells (TAG-72 positive). When the tumors grew to approximately 150-200 mm³, two doses of 5×10⁶ T cells were adoptively transferred by intravenous injection at five-day intervals. The values and error bars represent mean tumor size (mm³ f SEM). NT: non-transduced T cells; TAG-72 CAR-T: T cells transduced with a TAG-72 CAR; TAG-72 CAR/PD-1 KO T: PD-1 gene knock-out TAG-72 CAR-T cells; mean±SEM; representative data of the T cells from one donor are shown.

FIG. 7. Anti-tumor activity of RC3H1 and/or RC3H2 gene knock-out CAR-T cells in OVCAR-3 ovarian tumor NSG mice xenograft models. Four NSG mice per group were subcutaneously administered 1×10⁷ OVCAR-3 tumor cells (TAG-72 positive). When the tumors grew to approximately 150-200 mm³, two doses of 5×10⁶ T cells were adoptively transferred by intravenous injection at five-day intervals. The values and error bars represent mean tumor size (mm³ f SEM). NT: Non-transduced T cells; TAG-72 CAR-T: T cells transduced with a TAG-72 CAR TAG-72 CAR/RC3H1 KO T: RC3H1 gene knock-out TAG-72 CAR-T cells; TAG-72 CAR/RC3H2 KO T: RC3H2 gene knock-out TAG-72 CAR-T cells; TAG-72 CAR/RC3H1,2 KO T: RC3H1 and RC3H2 double gene knock-out TAG-72 CAR-T cells. **p<0.01, mixed-effect analysis with Greisser-Greenhouse correction and Dunnett's multiple comparison one-way ANOVA test comparing all group means against the TAG-72 CAR-T control group. Representative data of the T cells from one donor are shown.

FIG. 8. Anti-tumor activity of A2AR and FAS gene knock-out CAR-T cells in OVCAR-3 ovarian tumor NSG mice xenograft models. Four NSG mice per group were subcutaneously administered 1×10⁷ OVCAR-3 tumor cells (TAG-72 positive). When the tumors grew to approximately 150-200 mm³, two doses of 5×10⁶ T cells were adoptively transferred by intravenous injection at five-day intervals. The values and error bars represent mean tumor size (mm³ t SEM). NT: non-transduced T cells; TAG-72 CAR-T: T cells transduced with a TAG-72 CAR; TAG-72 CAR/A2AR KO T: A2AR gene knock-out TAG-72 CAR-T cells; TAG-72 CAR/FAS KO T: FAS gene knock-out TAG-72 CAR-T cells. *p<0.05, **p<0.01, #p<0.001, two-way ANOVA followed by Dunnett's multiple comparison test comparing all group means against the CAR-T control group. Representative data of the T cells from one donor are shown.

FIG. 9. Anti-tumor activity of TGFBR1 and TGFBR2 dominant negative gene mutation CAR-T cells in OVCAR-3 ovarian tumor NSG mice xenograft models. Four NSG mice per group were subcutaneously administered 1×10⁷ OVCAR-3 tumor cells (TAG-72 positive). When the tumors grew to approximately 150-200 mm³, two doses of 5×10⁶ T cells were adoptively transferred by intravenous injection at five-day intervals. The values and error bars represent mean tumor size (mm³±SEM). NT: Non-transduced T cells, TAG-72 CAR-T: T cells transduced with a TAG-72 CAR, TAG-72 CAR/TGFBR1 KO T: TGFBR1 dominant negative gene knock-out TAG-72 CAR-T cells, TAG-72 CAR/TGFBR2 KO T: TGFBR2 dominant negative gene knock-out TAG-72 CAR-T cells. *p<0.05, **p<0.01, ***p<0.001, two-way ANOVA followed by Dunnett's multiple comparison test comparing all group means against the CAR-T control group. Representative data of the T cells from one donor are shown.

FIG. 10. Anti-tumor activity of CD19 CAR-T cells with RC3H1 and/or RC3H2 gene knock-out in Raji lymphoma tumor NSG mice xenograft models. Four NSG mice per group were subcutaneously administered Raji tumor cells (CD19 positive). Three days after tumor inoculation, mice were treated with a single dose of 5×10⁶ CAR-T cells by intravenous injection. (A) Tumor size was monitored for 23 days. The values and error bars represent mean tumor size (mm³ f SEM). Multiple t-tests with Holm-Sidak correction were performed to compare RC3H1 and/or RC3H2 gene knock-out CD19 CAR-T cell groups with non-transfected CD19 CAR-T cells. (*p<0.005; **p<0.00l) (B) Kaplan-Meier survival curves were analysed using the Log-rank (Mantel-Cox) test. NT: Non-transduced T cells; CD19 CAR: CD19 CAR-T cells; CD19 CAR/RC3H1 KO: RC3H1 gene knock-out CD19 CAR-T cells; CD19 CAR/RC3H2 KO: RC3H2 gene knock-out CD19 CAR-T cells; CD19 CAR/RC3H1,2 KO: RC3H1 and RC3H2 double gene knock-out CD19 CAR-T cells. Representative data of the T cells from one donor are shown.

FIG. 11. Expression of activation markers on CD19 CAR-T cells with or without RC3H1 and/or RC3H2 gene KO after continued activation exposure. Graph shows the expression of activation markers CD25 and CD69 on CAR+ cells following 7 days of antigen exposure. CD19 CAR-T cells were generated from a single healthy donor. Results represent the average±SD of technical duplicates.

FIG. 12. CRISPR knock-out analysis of RC3H1 and RC3H2 gene in single and double knock-out T cells. RC3H1 and RC3H2 guide RNA formed RNPs were transfected into human activated T cells to generate RC3H1 or RC3H2 single KO (RC3H1 KO T cells or RC3H2 KO T cells), or RC3H1 and RC3H2 double KO T cells (RC3H1,2 KO T cells). Knock-out efficiencies were analysed using ICE analysis. Out-of-frame indel percentage is the proportion of indels that indicate a frameshift or are more than 21 bp in length.

FIG. 13. Effect of the RC3H1 and/or RC3H2 KO on the function of a T cell (CD8+, CD4+) with no CAR. T cells f RC3H1 and/or RC3H2 KOs were maintained in the presence of Dynabeads™ Human T-Activator CD3/CD28 beads (Thermofisher, Massachusetts, United States) (DB) for at least 92 h in T cell expansion media at a bead to cell ratio of 1:1. Beads were magnetically removed before using effector cells in an xCELLigence® assay. Effector cells were added to target cancer cells (in this instance, OVCAR-3) at an effector to target (E:T) ratio of 1:1. NCI was monitored over 20 h. Target cell elimination (observed as a reduction in NCI) was seen across all conditions. Importantly, following continued CD3/CD28 mediated activation, cells with genetically deleted RC3H1 and/or RC3H2 genes were able to more effectively eliminate target cells in vitro. Results represent the average±SEM of biological and intra-assay triplicate.

FIG. 14. CRISPR knock-out analysis of RC3H1 and RC3H2 gene in single and double knock-out NK-92 cells. RC3H1 and RC3H2 guide RNAs formed RNPs were transfected into NK-92 cells to generate RC3H1 or RC3H2 single KO (RC3H1 KO NK-92 cells or RC3H2 KO NK-92), or RC3H1 and RC3H2 double KO NK-92 cells (RC3H1,2 KO NK-92). Knock-out efficiencies were analyzed using ICE analysis. Out-of-frame indel percentage is the proportion of indels that indicate a frameshift or are more than 21 bp in length. R² value computed by Pearson correlation coefficient indicates the confidence of the indel percentage.

FIG. 15. Effect of the RC3H1 and/or RC3H2 KO on the function of NK-92 cells (with and without TAG-72 CAR). The ability for the NK cell line, NK-92±RC3H1 KO (green) or RC3H2 KO (purple) or RC3H1,2 KO (orange)±TAG-72 CAR to eliminate cancer cells in vitro was assessed using the real time cell monitoring system, xCELLigence®. (A) RC3H1 and/or RC3H2 gene(s) were deleted in the NK-92 cell line using CRISPR/Cas9. Resultant RC3H1 and/or RC3H2 KO NK-92 effector cells were added to target cancer cells (MES-OV (left panel) or OVCAR-3 (right panel) at an E:T ratio of 1:1. NCI was monitored over 40 h. Target cell elimination (observed as a reduction in NCI compared to target cells alone (blue)) was observed across all conditions. Results represent the mean±SEM of technical triplicates. (B) Further genetic manipulation of NK-92 cells was conducted to introduce a TAG-72 CAR. Lentivirus transduction was performed following transfection. Transduction efficiency was assessed by flow cytometry following ˜72 hrs in culture where values embedded within each dotplot represent proportion of CAR+ cells as a frequency of viable, single cells. Resultant TAG-72 CAR/RC3H1 and/or RC3H2 KO NK-92 cells were isolated using fluorescent activated cell sorting and their in vitro function was assessed as previously described. (C) NCI was monitored over 40 h. Results represent the mean±SEM, n=1-3.

FIG. 16. Generation of CRISPR gene knock-out induced pluripotent stem cells (iPSCs) as a source of cells for adoptive cell therapy. Workflow of deriving gene knock-out immune cells from iPSCs. iPSCs are transfected to knock-out the gene of interest. These cells are then sequenced to characterize and verify the knock-out, then differentiated into CD34+ cells and into immune cells.

FIGS. 17A-17B. RC3H1 and RC3H2 double KO in iPSC (RC3H1,2 KO iPSC) does not affect pluripotency. This was characterised by (A) morphology (scale bars=200 μm) where no differentiated cells are present and (B) flow cytometry analysis for iPSC markers TRA-1-60, TRA-1-81 and SSEA-4. Dead cells, debris and doublets were gated out, such that the histogram plots show all live cells in culture from either the non-transfected iPSC or RC3H1,2 KO iPSC samples. Greater than 99% of all live cells express all iPSC markers.

FIGS. 18A-18C. Transfection of RC3H1 and RC3H2 guide RNA formed RNP introduces insertions and deletions (indels) into the open reading frame of the specific genes in iPSCs. Sanger sequencing trace from the RC3H1 and RC3H2 gRNAs co-transfected iPSCs (“edited sample”) shows a heterogeneous mix of bases downstream of the cut site of RC3H1 gene (A) and RC3H2 gene (B) in contrast to the non-transfected iPSCs (“control sample”) (in A, SEQ ID NO: 27 sets forth 184 to 249 bp from the edited sample, SEQ ID NO: 28 sets forth 183 to 248 bp from the control sample; in B, SEQ ID NO: 29 sets forth 270 to 336 bp from the edited sample, SEQ ID NO: 30 sets forth 272 to 337 bp from the control sample). The black underlined region of the control sample represents the guide sequence and the horizontal red dotted underlined region is the associated PAM site. The vertical black dotted line on both traces represents the cut site. (C) CRISPR knock-out analysis of RC3H1 and RC3H2 gRNAs co-transfected iPSCs. Knock-out efficiency of RC3H1 and RC3H2 genes was assessed using ICE analysis. Out-of-frame indel percentage is the proportion of indels that indicate a frameshift or are more than 21 bp in length. R² value computed by Pearson correlation coefficient indicates the confidence of the indel percentage.

FIG. 19. RC3H1 and RC3H2 double KO in iPSC does not block differentiation toward iCD34+ cells. Unstained cells and cells stained with antibodies against CD34+ were analysed by flow cytometry. Dead cells, debris and doublets were gated out, such that the histogram plots show all live CD34+ cells in culture from either the non-transfected iPSC or RC3H1,2 KO iPSC samples. Deletion of both RC3H1 and RCH32 genes did not prevent iPSC development into subsets of iCD34 cells.

FIG. 20. iPSC containing RC3H1 and RC3H2 double KO are able to differentiate towards CD56+ cells with NK cytotoxic receptor expression of NKG2D and NKp46. Dead cells, debris and doublets were gated out, such that the CD56+ histograms show all live cells in culture generated. NKp46 and NKG2D plots were gated off CD56+ cells. Unstained and isotype controls are presented to show positive staining of each antibody for each respective receptor. The co-expression of NK functional receptors (NKp46 or NKG2D) with CD56 indicates that the CD56+ cells derived from RC3H1,2 KO iPSCs have the potential to perform NK-mediated cytotoxic function.

FIGS. 21A-21B. A2AR KO in iPSC does not affect pluripotency. This was characterised by (A) morphology (scale bars=200 μm) where no differentiated cells are present and (B) flow cytometry analysis for iPSC markers TRA-1-60, TRA-1-81 and SSEA-4. Dead cells, debris and doublets were gated out, such that the histogram plots show all live cells in culture from either the non-transfected iPSC or A2AR KO iPSC samples. Greater than 95% of all live cells express all iPSC markers.

FIG. 22A-22C. Transfection of A2AR guide RNAs formed RNP introduces insertions and deletions (indels) into the open reading frame of the A2AR gene in iPSCs. Frequency of indels was assessed by ICE analysis. (A) Sanger sequencing trace from the A2AR KO iPSCs (“edited sample”) shows a heterogeneous mix of bases downstream of the cut site in contrast to the non-transfected iPSCs (“control sample”). SEQ ID NO: 31 sets forth 134 to 199 bp from the edited sample; SEQ ID NO: 32 sets forth 137 to 202 bp from the control sample. The black underlined region of the control sample represents the guide sequence and the horizontal red dotted underlined region is the associated PAM site. The vertical black dotted line on both traces represents the cut site. (B) Relative percentage of the contribution of each edited sequence (normalized) in the genomic DNA from A2AR KO iPSCs. The sequences from top to bottom are set forth in SEQ ID NO: 33, 34, 35, 36, 37, and 38, respectively. (C) Distribution of the indel sizes in the entire edited population of RNP transfected iPSCs. Out-of-frame indel percentage is the proportion of indels that indicate a frameshift or are more than 21 bp in length. R² value computed by Pearson correlation coefficient indicates the confidence of the indel percentage.

FIG. 23. The inclusion of A2AR KO in iPSC does not block its differentiation toward iCD34+ cells. Cells stained with antibodies against CD34 were analyzed by flow cytometry. Unstained cells and cells stained with isotype controls were included as a control. Dead cells, debris and doublets were gated out, such that the histogram plots show all live cells in culture generated from either the non-transfected iPSC or A2AR KO iPSC samples. The inclusion of the KO does not block development of subtypes of iCD34+ cells.

FIG. 24. A2AR KO iPSCs are able to differentiate to iNK cells. Unstained cells and cells stained with antibodies against NK cell markers were analysed by flow cytometry. Dead cells, debris and doublets were gated out, such that the CD56+ histograms show all live cells in culture generated from either the non-transfected iPSC or A2AR KO iPSC samples. Unstained samples are presented to show clear positive staining of each antibody for each respective receptor. Appropriate isotype controls were also run and were negative. The expression of NK functional receptors (NKp46, NKp30, NKp44 and NKG2D) demonstrate that the CD56+ cells derived from A2AR KO iPSCs are iNK cells and are potentially capable of cytotoxic function.

FIG. 25. A2AR KO iPSCs are able to differentiate to functional iNK cells with enhanced in vitro killing activity. iNK cells were derived from non-transfected iPSC and A2AR KO iPSC. The function of resultant iNK cells was assessed in vitro using the real time cell monitoring system (xCELLigence®) where OVCAR-3 cells were used as targets. An effector to target ratio of 1:2 was used. (A) Change in NCI was recorded at 15 min intervals over at least 10 h of co-culture where a reduction in NCI is indicative of target cell death. (B) The results from (A) shown as % cytotoxicity of iNK cells relative to target cells alone controls at both 5 h (left panel) and 10 h (right panel) of co-culture. Cells were derived from a single iNK differentiation. Each data point represents technical replicates.

### DETAILED DESCRIPTION

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Through the specification and claims the terms “a” and “an” are to be taken to mean “at least one” and are not to be taken as excluding “two or more” unless the context clearly dictates otherwise.

A “nucleic acid construct”, as used herein, generally refers to a nucleic acid molecule that is constructed or made artificially or recombinantly, and is also interchangeably referred to as a nucleic acid vector. For example, a nucleic acid construct can be made to include a nucleotide sequence of interest that is desired to be transcribed in a cell, and in some instances, to produce an RNA molecule of a desired function (e.g., an antisense RNA, siRNA, miRNA, or gRNA), and in other instances, to produce an mRNA which is translated into a protein of interest (e.g., a Cas protein). The nucleotide sequence of interest in a nucleic acid construct can be operably linked to a 5′ regulatory region (e.g., a promoter such as a heterologous promoter), and/or a 3′ regulatory region (e.g., a 3′ untranslated region (UTR) such as a heterologous 3′ UTR). The nucleic acid construct can be in a circular (e.g., a plasmid) or linear form, can be an integrative nucleic acid (i.e., capable of being integrated into the chromosome of a host cell, e.g., a viral vector such as a lentiviral vector) or can remain episomal (e.g., a plasmid).

### General Description

Disclosed herein are methods of providing immune cells having enhanced function by inhibiting the function of one or more selected genes. For example, it has been demonstrated herein that ablation of one or more selected genes using CRISPR/Cas9 gene editing technology enhanced the persistence and anti-tumor activity of cytotoxic lymphocytes in vivo. Accordingly, methods are provided by inhibiting the function of one or more selected genes in immune cells, or in stem cells capable of differentiating into immune cells. Also disclosed herein are immune cells or stem cells made by the present methods, as well as the use of immune cells in therapeutic treatment.

### Immune Cells

An “immune cell”, as used herein, should be understood to include a cell of the mammalian immune system, for example, lymphocytes (T cells, B cells, NK cells and NKT cells), neutrophils, and monocytes (including macrophages and dendritic cells), and a cell line derived from cells of the mammalian immune system. An immune cell can be isolated from a mammalian subject, collected from a culture of cell line derived from an immune cell of a mammalian subject, or produced by differentiation from a stem cell.

This disclosure is directed to providing immune cells having enhanced function. By “enhanced function”, it is meant that an immune cell provided as a result of modification or manipulation disclosed herein, displays an enhanced activity (e.g., cytotoxicity), proliferation, survival, persistence, and/or infiltration, as compared to a control immune cell (i.e., an immune cell without the modification or manipulation). Cytotoxicity of an immune cell refers to the ability of an immune cell to kill a target cell, generally through a receptor-based mechanism.

In some embodiments, an immune cell is a cytotoxic immune cell, e.g., a cytotoxic lymphocyte.

In some embodiments, an immune cell is a T cell. In some embodiments, the T cell is an NKT cell. In some embodiments, an immune cell is a NK cell.

Reference to a “T cell” should be understood as a reference to any cell comprising a T cell receptor. In this regard, the T cell receptor may comprise any one or more of the α, β γ or δ chains. As would be understood by the person of skill in the art, NKT cells also express a T cell receptor and therefore target antigen specific NKT cells can also be generated according to the present invention. The present invention is not intended to be limited to any particular sub-class of T cell, although in one embodiment the subject T cell expresses an α/β TCR dimer. In some embodiments, said T cell is a CD4+ helper T cell, a CD8+ killer T cell, or an NKT cell. Without limiting the present invention to any one theory or mode of action, CD8+ T cells are also known as cytotoxic cells. As a major part of the adaptive immune system, CD8+ T cells scan the intracellular environment in order to target and destroy, primarily, infected cells. Small peptide fragments, derived from intracellular content, are processed and transported to the cell surface where they are presented in the context of MHC class I molecules. However, beyond just responding to viral infections, CD8+ T cells also provide an additional level of immune surveillance by monitoring for and removing damaged or abnormal cells, including cancers. CD8+ T cell recognition of an MHC I presented peptide usually leads to either the release of cytotoxic granules or lymphokines or the activation of apoptotic pathways via the FAS/FASL interaction to destroy the subject cell. CD4+ T cells, on the other hand, generally recognise peptide presented by antigen presenting cells in the context of MHC class II, leading to the release of cytokines designed to regulate the B cell and/or CD8+ T cell immune responses. CD4+ T cells with cytotoxic activity have also been observed in in various immune responses. Moreover, CD4+ CAR-T cells demonstrate equivalent cytotoxicity to CD8+ CAR-T cells in vitro, and even outperformed CD8+ CAR-T cells in vivo for longer antitumor activity (see, e.g., Wang et al., JCI Insight. 2018; 3(10):e99048; Yang et al., Sci Transl Med. 2017 Nov. 22; 9(417), eaag1209).

Natural killer T cells (also called NKT or T/NK cells) are a specialised population of T cells that express a semi-invariant T cell receptor (TCR α-β) and surface antigens typically associated with natural killer cells. The TCR on NKT cells is unique in that it commonly recognizes glycolipid antigens presented by the MHC I-like molecule CD1d. Most NKT cells express an invariant TCR alpha chain and one of a small number of TCR beta chains. The TCRs present on type I NKT cells commonly recognise the antigen alpha-galactosylceramide (alpha-GalCer). Within this group, distinguishable subpopulations have been identified, including CD4⁺CD8⁻ cells, CD4⁻ CD8⁺ cells and CD4⁻CD8⁻ cells. Type II NKT cells (or noninvariant NKT cells) express a wider range of TCR α chains and do not recognise the alpha-GalCer antigen. NKT cells produce cytokines with multiple, often opposing effects, for example either promoting inflammation or inducing immune suppression including tolerance. As a result, they can contribute to antibacterial and antiviral immune responses, promote tumor-related immunosurveillance, and inhibit or promote the development of autoimmune diseases. Like natural killer cells, NKT cells can also induce perforin-, FAS-, and TNF-related cytotoxicity. Accordingly, reference to T cells should be understood to include reference to NKT cells.

Natural killer (NK) cells are a type of cytotoxic lymphocyte that forms part of the innate immune system. NK cells provide rapid responses to virus-infected cells, acting at around 3 days after infection, and also respond to tumor formation. Typically, immune cells such as T cells detect major histocompatibility complex (MHC) presented on infected or transformed cell surfaces, triggering cytokine release and resulting in lysis or apoptosis of the target cell. NK cells, however, have the ability to recognize stressed cells in the absence of antibodies or MHC, allowing for a much faster immune reaction. This role is especially important because harmful cells that are missing MHC I markers cannot be detected and destroyed by other immune cells, such as T cells. In contrast to NKT cells, NK cells do not express TCR or CD3 but they usually express the surface markers CD16 (FcγRIII) and CD56.

In some embodiments, the immune cells to be modified or manipulated in accordance with the present methods can be isolated from a mammalian subject, including, e.g., blood (whole blood, serum or plasma), bone marrow, thymus, lymph node.

In some embodiments, the immune cells to be modified or manipulated in accordance with the present methods can be collected from a culture of cell line derived from an immune cell of a mammalian subject, e.g., T cell lines.

In some embodiments, the immune cells to be modified or manipulated in accordance with the present methods can be differentiated from a stem cell or other progenitor cells (such as cells cultured and differentiated from a stem cell). Methods for differentiating a stem cell into immune cells, in particular into T cells or NK cells, are known in the art (Li et al., Human iPSC-Derived Natural Killer Cells Engineered with Chimeric Antigen Receptors Enhance Anti-tumor Activity, Cell Stem Cell, 2018, 23(2): 181-192 e5; Themeli et al., Generation of tumor-targeted human T lymphocytes from induced pluripotent stem cells for cancer therapy, Nat Biotechnol, 2013, 31(10): 928-33; Maeda et al., Regeneration of CD8alphabeta T Cells from T-cell-Derived iPSC Imparts Potent Tumor Antigen-Specific Cytotoxicity, Cancer Res, 2016, 76(23): 6839-6850).

### Stem Cells

A “source cell”, as used herein, refers to the cell to be converted to a “derived cell” by reprogramming or differentiation. Examples of source cells suitable for use in the methods disclosed herein include stem cells. Examples of “derived cell” include immune cells such as T cells, NKT cells and NK cells.

The term “stem cell” should be understood as a reference to any cell which are capable of self renewal and exhibits the potential to develop in the direction of multiple lineages, given its particular phenotype, and thus to form a new organism or to regenerate a tissue or cellular population of an organism. The stem cells which are utilized in accordance with the present invention are pluripotent and multipotent and capable of differentiating along two or more lineages and include, but are not limited to, embryonic stem cells (ESCs), adult stem cells, umbilical cord stem cells, haemopoietic stem cells (HSCs), progenitor cells, precursor cells, pluripotent cells, multipotent cells or de-differentiated somatic cells (such as an induced pluripotent stem cell). By “pluripotent” is meant that the subject stem cell can differentiate to form, inter alia, cells of any one of the three germ layers, these being the ectoderm, endoderm and mesoderm.

In some embodiments, the source cell, also expresses at least one homozygous major HLA genotype. In some embodiments, a source cell expresses at least one homozygous HLA genotype which is a major transplantation antigen and which is preferably expressed by a significant proportion of the population, such as at least 5%, at least 10%, at least 15%, at least 17%, at least 20%, or more of the population. Where the homozygous HLA genotype corresponds to a dominant MHC I or MHC II HLA type (in terms of tissue rejection), the use of such a cell will result in significantly reduced problems with tissue rejection in the wider population who receive the cells of the present invention in the context of a treatment regime. In other embodiments, a source cell may be homozygous in relation to more than one HLA antigen, e.g., two, three, or more HLA antigens. HLA antigens of interest can be selected from e.g., HLA A1, B8, C7, DR17, DQ2, or HLA A2, B44, C5, DR4, DQ8, or HLA A3, B7, C7, DR15, DQ6.

In some embodiments, the source cell is homozygous in relation to the inhibited gene.

In some embodiments, a source cell has been genetically modified in one or more genes identified herein so that the function of the modified gene(s) in a derived cell differentiated from the genetically modified source cell is inhibited.

In some embodiments, a source cell has also been genetically modified to comprise a nucleic acid encoding a CAR (i.e., a chimeric antigen receptor). Nucleic acids encoding CARs can be introduced into a source cell by methods known in the art.

In some embodiments, a source cell is a stem cell. In some embodiments the source cell is an induced pluripotent stem cell (iPSC).

In some embodiments, progenitor cells capable of differentiating into an immune cell are used to be modified; for example, cells cultured from a pluripotent stem cell (such as an iPSC), which have undergone some differentiation in the culture towards an immune cell, but have not fully differentiated into an immune cell.

iPSC

iPSCs are usually generated directly from somatic cells. iPSC can be induced in principle from any nucleated cell including, for example, mononucleocytes from blood and skin cells. In some embodiments, iPSCs may be generated from fully differentiated T cells; or from precursor T cells, such as thymocytes, which precursor T cells have begun or even completed the re-arrangement of their TCRs and exhibit an antigen specificity of interest. In another embodiment, an iPSC is transfected with one or more nucleic acid molecules coding for a TCR (such as rearranged TCR genes) directed to an antigenic determinant of interest (e.g., a tumour antigenic determinant). In one embodiment, an iPSC is derived from a cell which expresses a rearranged TCR, preferably a rearranged αβ TCR In another embodiment, said cell expresses a rearranged γδ TCR. Examples of cells suitable for use in generating the iPSCs of the present invention include, but are not limited to CD4+ T cells, CD8+ T cells, NKT cells, thymocytes or other form of precursor T cells.

In another embodiment iPSCs is derived from another type of immune cell such as NK cells.

Methods for generating iPSCs from mature or differentiated cells (such as T cells or precursor T cells) are known to the person of skill in the art (Themeli, Kloss et al. 2013, Li, Hermanson et al. 2018).

In some embodiments, a source cell is an induced pluripotent stem cell (iPSC).

In some embodiments, a source cell is generated from cord blood PBMC (peripheral blood mononuclear cell).

In some embodiments, the subject source cell is a cell that is more differentiated towards an immune cell as compared to a pluripotent stem cell.

Derived immune cells generated by the methods disclosed herein include hematopoietic lineage cells capable of differentiating into an immune cell, and particular types of immune cells. Examples of derived immune cells are HE, pre-HSC, HSC, multipotent progenitor cells, common lymphoid progenitor cells, early thymic progenitor cells, pre-T cell progenitor cells, pre-NK progenitor cells, T progenitor cells, NK progenitor cells, macrophages and other immune cells such as T cells, NK-T cells and NK cells.

This disclosure is directed to providing immune cells or derived immune cells produced by differentiation having enhanced function. By “enhanced function” it is meant that an immune cell, provided as a result of modification or manipulation disclosed herein, displays an enhanced activity (e.g., cytotoxicity), proliferation, survival, persistence, and/or infiltration, as compared to a control immune cell (i.e., an immune cell without the modification or manipulation). Cytotoxicity of an immune cell refers to the ability of an immune cell to kill a target cell, generally through a receptor-based mechanism.

### Genes to be Inhibited

In accordance with this disclosure, inhibition of the function of one or more genes identified herein can enhance the function of an immune cell.

By “inhibition of the function of a gene” as used herein, it is meant that the level and/or activity of the protein encoded by the gene is ultimately reduced or eliminated. Thus, the function of a gene can be inhibited as a result of manipulation or modification to the genomic DNA sequence of the gene (e.g., leading to a disruption of the gene), as a result of inhibiting the mRNA (e.g., reducing the level or function of the mRNA, e.g., by inhibiting transcription or translation), or as a result of inhibiting the protein (e.g., by reducing the level or activity of the protein). In some embodiments, the extent of inhibition is at least 20%, 30%, 40%, 50%, 60%, 70%, 80%, 90%, 95% or more, when the level and/or activity of the protein encoded by a gene in a modified cell is compared to the level and/or activity of the protein in an unmodified cell.

In some embodiments, the gene whose function is to be inhibited is selected from the group consisting of RC3H1, RC3H2, A2AR, FAS, TGFBR1, and TGFBR2. In some embodiments, inhibition is directed to a single gene selected from the group consisting of RC3H1, RC3H2, A2AR, FAS, TGFBR1, and TGFBR2; e.g., a single gene that is RC3H1, RC3H2, A2AR, FAS, TGFBR1, or TGFBR2. In some embodiments, inhibition is directed to a single gene selected from the group consisting of RC3H1, RC3H2, A2AR, FAS, TGFBR1, and TGFBR2, in combination with inhibition of at least another gene. In some embodiments, inhibition is directed to two or more of the genes selected from the group consisting of RC3H1, RC3H2, A2AR, FAS, TGFBR1, and TGFBR2, e.g., the RC3H1 and RC3H2 genes, the TGFBR1 and TGFBR2 genes, the TGFBR1 and RC3H2 genes; and optionally in combination with inhibition of at least another gene.

As described below, members of the group of genes consisting of RC3H1, RC3H2, A2AR, FAS, TGFBR1, and TGFBR2 are known in the art as being implicated in immune cell function. However, it is not known in the art whether inhibition of the function of these genes, individually or in combination, may have adverse consequences. In particular, completely removing the function of these genes (for example, by gene editing) could be anticipated to adversely affect important cell functions, thereby resulting in cells with reduced viability or ability to replicate. Further, removing the function of these genes in stem cells (for example, iPSCs) could be anticipated to adversely impact cell functions such as viability, self-renewal, pluripotency, ability to differentiate into particular cell types (for example, immune cells) and for those cell types to be functional. It will be recognised by a person skilled in the art that maintenance of these critical cell functions is a critical feature of the present invention.

### RC3H1, RC3H2

RC3H1 is also known as RC3H1, Roquin-1, Ring Finger And CCCH-Type Domains 1, RING Finger And CCCH-Type Zinc Finger Domain-Containing Protein 1, RING Finger and C3H Zinc Finger Protein 1, Ring Finger And CCCH-Type Zinc Finger Domains 1, ROQ1, RNF198, or RING Finger Protein 198.

RC3H2 is also known as Roquin-2, Roquin2, Ring finger And CCCH-type domains 2, Ring Finger And CCCH-Type Zinc Finger Domain-Containing Protein 2, Ring Finger And CCCH-Type Zinc Finger Domains 2, MNAB, ROQ2, RNF164, or RING Finger Protein 164.

The ROQUIN family of proteins includes ROQUIN1 (encoded by RC3H1) and ROQUIN2 (encoded by RC3H2), which are RNA biding proteins that play important roles in both innate and adaptive immune systems (Athanasopoulos, V., R. R. Ramiscal, and C. G. Vinuesa, ROQUIN signalling pathways in innate and adaptive immunity. Eur J Immunol, 2016, 46(5): p. 1082-90). A Rc3 h1 mutation in mice (sanroque mice) results in increased ICOS expression in T cells, which causes lupus-like auto-immune syndrome in mice (Yu, D., et al., Roquin represses autoimmunity by limiting inducible T-cell co-stimulator messenger RNA. Nature, 2007, 450(7167): p. 299-303). Though RC3H1 or RC3H2 knock-out alone in mice did not develop autoantibodies and lacked autoimmunity, RC3H1 and RC3H2 double knock-out mice showed similar immunophysiologic phenotype of sanroque mice. No humans have been found to carry disease causing mutations in RC3H1 or RC3H2 to date (Athanasopoulos, V., RR. Ramiscal, and C. G. Vinuesa, ROQUIN signalling pathways in innate and adaptive immunity. Eur J Immunol, 2016, 46(5): p. 1082-90). The role of RC3H1 and RC3H2 genes in human T cells, especially its function in cytotoxic cells, was unknown prior to this disclosure.

In accordance with this disclosure, inhibition of the function of either or both of RC3H1 and RC3H2 genes enhances the function of an immune cell.

### A2AR

A2AR is also known as ADORA2A, Adenosine A2a Receptor, Adenosine Receptor A2a, ADORA2, Adenosine Receptor Subtype A2a, or RDC8.

Extracellular adenosine generated by tumor cells is a key immunosuppressive metabolite that restricts activation of cytotoxic lymphocytes and inhibits antitumor immune responses through adenosine2A receptor (A2AR).

In accordance with this disclosure, inhibition of the function of the A2AR gene, e.g., through gene editing (e.g., mediated by CRISPR/Cas9 based on specifically designed guide RNAs), enhances the function of an immune cell.

### FAS

FAS is also known as Fas cell surface death receptor, APT1, CD95, FAS1, APO-1, FASTM, ALPS1A, or TNFRSF6.

The FAS receptor (also known as CD95 and APO-1) induces apoptosis and terminal differentiation of cytotoxic T cells. Engagement of FAS with its ligand FASL could possibly dampen the anti-tumor activity of CAR-T cells.

In accordance with this disclosure, inhibition of the function of the FAS gene, e.g., through gene editing (e.g., mediated by CRISPR/Cas9), enhances the function of an immune cell.

### TGFBR1 and TGFBR2

TGFBR1 is also known as TGFRBR1, TGFB receptor 1, TGF-β receptor 1, AAT5, ALK5, ESS1, LDS1, MSSE, SKR4, TBRI, ALK-5, LDS1A, LDS2A, TBR-I, TGFR-1, ACVRLK4, tbetaR-I, Transforming Growth Factor Beta Receptor 1, or Transforming Growth Factor Beta Receptor I.

TGFBR2 also known as TGFBRII, AAT3, FAA3, LDS2, MFS2, RIIC, LDS1B, LDS2B, TAAD2, TBRII, TBR-ii, TGFR-2, TGFbeta-RII, Transforming Growth Factor Beta Receptor 2, or Transforming Growth Factor Beta Receptor II.

TGF-β exerts systemic immune suppression and inhibits host immunosurveillance, and is considered to be one of the major factors of the immunosuppressive microenvironment in tumor.

In accordance with this disclosure, inhibition of the function of the TGFBR1 and/or TGFBR2 genes, e.g., through gene editing (e.g., mediated by CRISPR/Cas9 based on specifically designed guide RNAs), enhances the function of an immune cell.

In accordance with this disclosure, inhibition of the function of at least one of the genes selected from the group consisting of RC3H1, RC3H2, A2AR, FAS, TGFBR1, and TGFBR2, in combination with inhibition of at least another gene, enhances the function of an immune cell.

### Inhibiting the Function of a Gene

Inhibition of the function of a gene can be achieved by a variety of approaches, for example, through gene editing, inhibiting translation via, for example, RNA interference or antisense oligonucleotides, or through the use of compounds such as small molecules or antibodies that directly antagonize the protein product.

### Inhibiting Through Gene Editing

In some embodiments, inhibition of the function of a gene is achieved through the use of a gene editing system that modifies the genomic sequence of a gene.

A gene editing system typically involves a DNA-binding protein or DNA-binding nucleic acid, coupled with a nuclease. The DNA-binding protein or DNA-binding nucleic acid specifically binds to or hybridizes to a targeted region of a gene, and the nuclease makes one or more double-stranded breaks and/or one or more single-stranded breaks in the targeted region of the gene. The targeted region can be the coding region of the gene, e.g. in an exon, near the N-terminal portion of the coding region (e.g., in the first or second exon). The double-stranded or single-stranded breaks may undergo repair via a cellular repair process, such as by non-homologous end-joining (NHEJ) or homology-directed repair (HDR). In some instances, the repair process introduces insertion, deletion, missense mutation, or frameshift mutation (including, e.g., biallelic frameshift mutation), leading to disruption of the gene and inhibition of the function of the gene.

Examples of gene editing systems include a fusion comprising a DNA-binding protein and a nuclease, such as a Zinc Finger Nuclease (ZFN) or TAL-effector nuclease (TALEN), or an RNA-guided nuclease such as a clustered regularly interspersed short palindromic nucleic acid (CRISPR)-Cas system.

### ZFPs and TALENs

In some embodiments, inhibiting of the function of a gene is achieved by utilizing a gene editing system that includes a DNA-binding protein such as one or more zinc finger proteins (ZFP) or a transcription activator-like protein (TAL), fused to an endonuclease. Examples include ZFNs, TALEs, and TALENs.

The DNA binding domains of ZFPs and TAL can be “engineered” to bind to a target DNA sequence of interest. For example, one or more amino acids of the recognition helix region of a naturally occurring zinc finger or TALE protein can be modified so as to direct binding to a predetermined DNA sequence. Criteria for rational design are described, e.g., U.S. Pat. Nos. 6,140,081, 6,453,242, 6,534,261, WO 98/53058, WO 98/53059, WO 98/53060, WO 02/016536, WO 03/016496, and U.S. Publication No. 20110301073 A1.

In some embodiments, the DNA-binding protein comprises a zinc-finger protein (ZFP) or one or more zinc finger domains of a ZFP. ZFP or domains thereof bind to DNA in a sequence-specific manner through one or more “zinc fingers” (regions of amino acids within the binding domain whose structure is stabilized through coordination of a zinc ion). Sequence-specificity of a natural occurring ZFP can be altered by making amino acid substitutions at certain positions on a zinc finger recognition helix. In addition, many engineered, gene-specific zinc fingers are available commercially (see, e.g., the CompoZr platform for zinc-finger construction, developed by Sangamo Biosciences (Richmond, Calif., USA) in partnership with Sigma-Aldrich (St. Louis, Mo., USA)). Thus, in some embodiments, the ZFP is engineered to bind to a target sequence within a gene which is identified herein to be inhibited. Typical target sequences include exons, regions near the N-terminal region of the coding sequence (e.g., first exon, second exon), and the 5′ regulatory region (promoter or enhancer regions). A ZFP is fused to an endonuclease or a DNA cleavage domain to form a zinc-finger nuclease (ZFN). Examples of DNA cleavage domains include a DNA cleavage domain of a Type IIS restriction enzyme.

In some embodiments, a ZFN is introduced into a cell (e.g., an immune cell or a stem cell) via transfection of a nucleic acid construct (e.g., a plasmid, mRNA or viral vector) comprising a nucleic acid sequence encoding the ZFN. The ZFN is then expressed in the cell from the construct and leads to editing and disruption of a target gene. In some embodiments, a ZFN is introduced into a cell in its protein form.

In some embodiments, the DNA-binding protein comprises a naturally occurring or engineered transcription activator-like protein (TAL) DNA binding domain, such as in a transcription activator-like protein effector (TALE) protein. See, e.g., US 20110301073 A1, incorporated herein by reference. A TALE DNA binding domain is a polypeptide comprising one or more TALE repeats, with each repeat being 33-35 amino acids in length and including 1 or 2 DNA-binding residues. It has been determined that an HD (Histadine-Aspatate) sequence at positions 12 and 13 of a TAL repeat leads to a binding to cytosine (C), NG (Asparagine-Glycine) binds to T, NI (Asparagine-Isoleucine) to A, and NN (Asparagine-Asparagine) binds to G or A. See, e.g., US 20110301073 A1. In some embodiments, TALEs can be designed to have an array of TAL repeats with specificity to a target DNA sequence of interest within a gene identified herein to be inhibited. Custom-designed TALE arrays are also commercially available through Cellectis Bioresearch (Paris, France), Transposagen Biopharmaceuticals (Lexington, Ky., USA), and Life Technologies (Grand Island, N.Y., USA). In some embodiments, a TAL DNA binding domain is fused to an endonuclease to form a TALE-nuclease (TALEN), which cleaves a nucleotide sequence at a target site within a gene identified herein to be inhibited.

In some embodiments, a TALEN is introduced into a cell (e.g., an immune cell or a stem cell) via transfection of a nucleic acid construct (e.g., a plasmid, mRNA or viral vector) comprising a nucleic acid sequence encoding the TALEN. The TALEN is then expressed in the cell from the construct and leads to editing and disruption of a target gene. In some embodiments, a TALEN is introduced into a cell in its protein form.

### CRISPR/Cas

In some embodiments, inhibition of the function of a gene is achieved by utilizing a CRISPR (for “Clustered Regularly Interspaced Short Palindromic Repeats”)/Cas (for “CRISPR-associated nuclease”) system for gene editing. CRISPR/Cas is well known in the art with reagents and protocols readily available (Mali et al., 2013, Science, 339(6121), 823-826; Hsu et al., 2014, Cell, 157.6: 1262-1278; Jiang et al., 2013, Nature Biotechnology, 31, 233-239; Anzalone et al., Nature (2019) doi:10.1038/s41586-019-1711-4; Komor et al., Nature 533: 420-424, 2016; Gaudelli et al., Nature 551: 464-471 (2017)). Exemplary CRISPR-Cas gene editing protocols are described in Jennifer Doudna, and Prashant Mali, 2016, “CRISPR-Cas: A Laboratory Manual” (CSHL Press, ISBN: 978-1-621821-30-4) and Ran et al. 2013, Nature Protocols, 8 (11): 2281-2308.

A CRISPR/Cas system generally comprises two components: (1) an RNA-dependent DNA nuclease, also referred to herein as a CRISPR endonuclease or a Cas protein, such as Cas9, Cas12 or other alternative nucleases; and (2) a non-coding short “guide RNA” which comprises either a dual RNA comprising a crRNA (“CRISPR RNA”) and a tracrRNA (“transactivating crRNA”), or a single-chain full length guide RNA, and comprises a targeting sequence that directs the nuclease to a target site in the genome. The guide RNA (gRNA) directs the nuclease to the target site where the nuclease generates a double-stranded break (DSB) in the DNA at the target site. The resulting DSB is then repaired by one of two general repair pathways: the Non-Homologous End Joining (NHEJ) pathway and the Homology Directed Repair (HDR) pathway. The NHEJ repair pathway is the most active repair mechanism, capable of rapidly repairing DSBs, but frequently results in small nucleotide insertions or deletions (Indels) at the DSB site, resulting in a frameshift mutation to knock-out a functional gene. The HDR pathway is less efficient but with high-fidelity. When a CRISPR endonuclease is provided with a DNA template homologous to the break region, the double-stranded break is repaired using the homologous DNA template via HDR The HDR pathway allows insertion of large gene inserts into cells along with RNPs.

Design or selection of a gRNA sequence that comprises a sequence targeting a target site in a gene of interest has been described in the art. The target site can include sequences of regulatory regions (such as promoters and enhancers), or sequences within the coding region (such as exons, e.g., exons near the 5′ end, or an exon encoding a particular domain or region of the protein). In some embodiments, a target site is selected based on its location immediately 5′ of a PAM sequence, such as typically NGG, or NAG.

A guide sequence is designed to include a targeting sequence having complementarity with a target sequence (a nucleotide sequence at a target site). Full complementarity is not necessarily required, as long as there is sufficient complementarity to cause specific hybridization between a guide sequence and a target sequence and promote formation of a CRISPR complex at the target site. In some embodiments, the degree of complementarity between the targeting sequence of a gRNA and a target sequence is at least 80%, 85%, 90%, 95%, 98%, 99% or higher (e.g., 100% or fully complementary).

In some embodiments, a guide sequence is at least 15 nucleotides, e.g., 15, 16, 17, 18, 19, 20, 21, 22, 23, 24, 25, 26, 27, 28, 29, 30, 35, 40, 45, 50, 55, 60, 65, 70 or 75 or more, nucleotides in length. In some embodiments, a guide sequence is not more than 75, 70, 65, 60, 55, 50, 45, 40, 35, 30, 25, or 20 nucleotides in length. In some embodiments, the targeting sequence portion of a guide sequence is about 20 nucleotides in length. Truncated gRNAs, with shorter regions (<20 nucleotides) of target complementarity, have been described as effective with improved target specificity (see, e.g., Fu et al., Nature Biotechnol., 32(3): 279-284, 2014). Thus, in some embodiments, the targeting sequence of a guide RNA is 17, 18, 19 or 20 nucleotides in length. In some embodiments, the targeting sequence of a guide RNA is fully complementary to a nucleotide sequence at a target site. In some embodiments where the targeting sequence of a guide RNA is not fully complementary to a nucleotide sequence at a target site, the portion of the targeting sequence that is close to the PAM sequence in the genome (also referred to as the seed region) is fully complementary to a nucleotide sequence at a target site. In other words, some variation in the nucleotides 5′ of the guide sequence (i.e., the non-seed region) is permissible. For example, a guide sequence can be designed to include a targeting portion of at least 17 nucleotides in length (e.g., 17, 18, 19 or 20 nucleotides in length), having a seed region of at least 17 nucleotides being fully complementary to at least 17 nucleotides in a target sequence.

Examples of target sequences in specific genes are provided in Table 1. In some embodiments, a guide sequence includes a targeting sequence of 17-20 nucleotides, with at least the 17 nucleotides in the seed region (the 3′ portion of the targeting sequence) being fully complementary to at least 17 nucleotides in a target sequence, e.g., to the 17 nucleotides from the 3′ end of a target sequence.

TABLE 1

| Gene | | | |
|---|---|---|---|
| Official | Genomic | | Target Sequences |
| Symbol | RefSeqGene | Target Transcript | (a nucleotide sequence at a target site) |
| PDCD1 | NG_012110.1 | ENSG00000188389 | GTCTGGGCGGTGCTACAACT (SEQ ID NO: 1) |
| RC3H1 | NC_000001.11 | ENSG00000135870 | TGCCTGTACAAGCTCCACAA (SEQ ID NO: 2) |
| | | | GAGAGGAAATCCGTCCATTG (SEQ ID NO: 3) |
| RC3H2 | NC_000009.12 | ENSG00000056586 | TGTGAACAACCTAAACTGAT (SEQ ID NO: 4) |
| | | | TGCCTGTGCAGGCAGCTCAA (SEQ ID NO: 5) |
| | | | AGCTTCCACAATGCCTGTGC (SEQ ID NO: 6) |
| A2AR | NG_052804.1 | ENSG00000128271 | CTCCACCGTGATGTACACCG (SEQ ID NO: 7) |
| | | | CTCCTCGGTGTACATCACGG (SEQ ID NO: 8) |
| FAS | NG_009089.2 | ENSG00000026103 | GTGACTGACATCAACTCCAA (SEQ ID NO: 9) |
| | | | GGAGTTGATGTCAGTCACTT (SEQ ID NO: 10) |
| TGFBR1 | | ENSG00000106799 | CTCGATGGTGAATGACAGTG (SEQ ID NO: 11) |
| | | | GGTGAATGACAGTGCGGTTG (SEQ ID NO: 12) |
| | | | CCATCGAGTGCCAAATGAAG (SEQ ID NO: 13) |
| TGFBR2 | | ENSG00000163513 | GCTTCTGCTGCCGGTTAACG (SEQ ID NO: 14) |
| | | | TTGAACTCAGCTTCTGCTGC (SEQ ID NO: 15) |
| | | | GCAGAAGCTGAGTTCAACCT (SEQ ID NO: 16) |

A gRNA database for CRISPR genome editing is publicly available, which provides exemplary sgRNA target sequences in constitutive exons of genes in the human genome or mouse genome (see, e.g., the gRNA-database provided by GenScript, and by Massachusetts Institute of Technology; see also, Sanjana et al. (2014) Nat. Methods, 11:783-4). In some embodiments, the gRNA sequence is or comprises a sequence with minimal off-target binding to a non-target gene.

Examples of Cas proteins or CRISPR endonucleases suitable for use herein include Cpf1 (Zetsche et al., Cell (2015) 163(3): 759-771), Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4, or a functional derivative thereof (i.e., a mutant form or a derivative of a naturally occurring CRISPR endonuclease, such as a fragment thereof, that substantially retains the RNA-dependent endonuclease activity of the naturally occurring form). See, e.g., US20180245091A1 and US20190247517A1. In some embodiments, a Cas protein is Cas9, e.g., Cas9 from S. pyogenes, S. aureus or S. pneumoniae. In some embodiments, the Cas protein is a Cas9 protein from S. pyogenes having the amino acid sequence provided in the SwissProt database under accession number Q99ZW2.

In some embodiments, inhibition of the function of a gene is achieved through CRISPR-mediated gene editing, which comprises introducing into a cell (e.g., an immune cell or a stem cell) a first nucleic acid encoding a Cas nuclease, and a second nucleic acid encoding a guide RNA (gRNA) specific to a target sequence in a gene identified herein to be inhibited. The two nucleic acids can be included in one nucleic acid construct (or vector), or provided on different constructs (or vectors), to achieve expression of the Cas protein and the gRNA in the cell. Expression of the Cas nuclease and the gRNA in the cell directs the formation of a CRISPR complex at the target sequence, which leads to DNA cleavage.

In some embodiments, inhibition of the function of a gene is achieved through CRISPR-mediated gene editing, which comprises introducing into a cell a combination or complex between a gRNA and a Cas nuclease. In some embodiments, a Cas protein/gRNA combination or complex can be delivered into a cell via e.g., electroporation, particle gun, calcium phosphate transfection, cell compression or squeezing, liposomes, nanoparticles, microinjection, naked DNA plasmid transfer, protein transduction domain mediated transduction or virus mediated (including integrating viral vectors such as retrovirus and lentivirus, and non-integrating viral vectors such as adenovirus, AAV, HSV, vaccinia).

Regardless of the specific gene editing method used, in order to confirm that a gene sequence has been modified and the gene function has been inhibited, a variety of assays may be performed, including for example, by examining the DNA or mRNA via Southern and Northern blotting, PCR including RT-PCR, or nucleic acid sequencing, or by detecting the presence or activity of a particular protein or peptide via, e.g., immunological means (ELISAs and Western blot).

In some embodiments, the function of at least one of the RC3H1, RC3H2, A2AR, and FAS genes is inhibited by introducing indel(s) into an early exon of at least one of these genes through a CRISPR/Cas9 system, which results in frame-shift mutation(s) in at least one of these gene such that no functional protein is translated from an edited gene. In some embodiments, the functions of two or more of the RC3H1, RC3H2, A2AR, and FAS genes are inhibited by introducing an indel into an early exon of the two or more of these genes using CRISPR/Cas9, resulting in a frame-shift mutation in two or more of these gene such that no functional protein is translated from an edited gene. In some embodiments, the two or more of the RC3H1, RC3H2, A2AR, and FAS genes comprise RC3H2, in combination with another gene, e.g., RC3H2 and RC3H1.

In some embodiments, the function of at least one of the TGFBR1 and TGFBR2 genes is inhibited by introducing an indel into an exon and upstream of the codon for the starting amino acid residue of the intracellular signal transduction domain of the at least one of these genes through a CRISPR/Cas9 system, resulting in a frame-shift mutation that removes the intracellular signal transduction domain, which is a dominant negative mutation. In some embodiments, the functions of both of the TGFBR1 and TGFBR2 genes are inhibited by introducing an indel into an exon and upstream of the codon for the starting amino acid residue of the intracellular signal transduction domain of each of these genes using CRISPR/Cas9, resulting in a frame-shift mutation that removes the intracellular signal transduction domain, which is a dominant negative mutation.

CRISPR/Cas system can also be used without double-strand breaks or donor DNA, by using Nickases (i.e., Cas9 nickase) and High Fidelity Enzymes. See, e.g., Anzalone, A et al., Nature (2019) doi:10.1038/s41586-019-1711-4; Komor et al., Nature 533: 420-424, 2016; Gaudelli et al., Nature 551: 464-471 (2017).

Inhibiting Through Reducing or Eliminating the Level or Function of mRNA

In some embodiments, inhibition of the function of a gene is achieved by reducing or eliminating the level or function of the mRNA transcribed from the gene, i.e., inhibition of the mRNA. Unlike inhibition through a gene editing system, inhibition of mRNA is transient.

In some embodiments, inhibition of mRNA can be achieved through the use of e.g., an antisense nucleic acid, a ribozyme, a small interfering RNA (siRNA), a short hairpin RNA (shRNA), a miRNA (microRNA) or a precursor thereof, or a nucleic acid construct that can be transcribed in a cell to produce an antisense RNA, an siRNA, an shRNA, a miRNA or a precursor thereof.

Antisense—Antisense technology is a well-known method. An antisense RNA is an RNA molecule that is complementary to the full length or a part of an endogenous mRNA and blocks translation from the endogenous mRNA by forming a duplex with the endogenous mRNA. An antisense RNA can be made synthetically and introduced into a cell of interest (e.g., an immune cell), or made in the cell of interest through transcription from an exogenously introduced nucleic acid construct, to achieve inhibition of expression of a gene of interest. It is not necessary for an antisense RNA to be complementary to the full-length mRNA from a gene of interest. However, an antisense RNA should be of a length sufficient for forming a duplex with the target mRNA and blocking translation based on the target mRNA. Typically, an antisense RNA is at least 15 nucleotides, e.g., 15, 16, 17, 18, 19, 20, 21, 22, 23, 24, 25, 26, 27, 30, 35, 40, 50, 75, 100, 200, 300, 400, 500 nucleotides or more in length. In some embodiments, an antisense RNA is not more than 500, 400, 300, 200, 100, 75 or 50 nucleotides in length. Antisense molecules can also be DNA, DNA analogs and RNA analogs.

Ribozyme—A ribozyme (i.e., catalytic RNA) can be designed to specifically pair with a target RNA and cleave the phosphodiester backbone at a specific location, thereby functionally inactivating the target RNA. See, e.g., U.S. Pat. Nos. 6,423,885, 5,254,678, and Perriman et al., PNAS 92(13):6175-6179 (1995). A ribozyme can be made synthetically and introduced into a cell of interest (e.g., an immune cell), or made in the cell of interest through transcription from an exogenously introduced nucleic acid construct.

RNAi (RNA Interference)—Inhibition of gene expression or translation through RNAi is known in the art and can be achieved utilizing RNA molecules such as an siRNA (for “small interfering RNA”), shRNA (for “short hairpin RNA”), and a miRNA (for “microRNA”). siRNAs and shRNAs are known to be involved in the RNA interference pathway and interfere with the expression of a specific gene. siRNAs are small (typically 20-25 nucleotides in length), double-stranded RNAs and can be designed to include a sequence homologous to or complementary with a target mRNA (i.e., the mRNA transcribed from a gene of interest) or a portion of a target mRNA. shRNAs are cleaved by ribonuclease DICER to produce siRNAs. Given the sequence of a target gene, siRNAs or shRNAs can be designed and made either synthetically and introduced into a cell of interest (e.g., an immune cell), or made in a cell of interest (e.g., an immune cell) from an exogenously introduced nucleic acid construct encoding such an RNA. miRNAs are also small RNA molecules (generally about 21-22 nucleotides) that are processed from long precursors transcribed from non-protein-encoding genes, and interrupt translation through imprecise base-pairing with target mRNAs. miRNA or a precursor thereof (pri-miRNA or pre-miRNA) can be made synthetically and introduced to a cell of interest (e.g., an immune cell), or made in a cell of interest (e.g., an immune cell) from an exogenously introduced nucleic acid construct encoding either the miRNA or a precursor thereof.

In some embodiments, inhibition of mRNA can be achieved using a modified version of a CRISPR/Cas system where a Cas molecule that is an enzymatically inactive nuclease is used in combination with a gRNA targeting a gene of interest. The target site can be in the 5′ regulatory region (e.g., the promoter or enhancer region) of the gene. In some embodiments, the Cas molecule is an enzymatically inactive Cas9 molecule, which comprises a mutation, e.g., a point mutation, that eliminates or substantially reduces the DNA cleavage activity (see e.g. WO2015/161276). In some embodiments, an enzymatically inactive Cas9 molecule is fused, directly or indirectly, to a transcription repressor protein.

### Inhibiting Through Other Means

The invention incudes other methods known in the art for inhibiting the function of a gene, including for reducing the level or activity of the protein encoded by the gene, e.g. by introducing into a cell (e.g., an immune cell) a compound (e.g., a small molecule, an antibody, among others) that directly inhibits the activity of the protein encoded by the gene.

### CAR

In some embodiments, a cell (e.g., an immune cell or a stem cell) that has been modified to have inhibition of one or more selected genes has also been modified to contain a nucleic acid encoding a chimeric antigen receptor (or “CAR”).

In some embodiments, a nucleic acid encoding a CAR can be introduced into a cell prior to, simultaneous with, or subsequent to, the cell being modified to inhibit the function of a selected gene. In embodiments where the inhibition is transient (e.g., through an antisense RNA or RNAi), a nucleic acid encoding a CAR is preferably introduced into a cell prior to the cell being modified to achieve inhibition. In embodiments where the inhibition is permanent (e.g., through gene editing), a nucleic acid encoding a CAR can be introduced into a cell prior to, simultaneous with, or subsequent to, the cell being modified to achieve inhibition. In some embodiments, a nucleic acid encoding a CAR is designed to allow insertion by HDR at the target site of gene editing following the introduction of the DSBs, i.e., the gene is disrupted by knock-in or insertion of the CAR-encoding nucleic acid.

In some embodiments, the CAR gene can be introduced into cells via multiple technologies, including lentiviral or retroviral vectors, transposon systems, CRISPR-Cas9 or TALEN mediated gene knock-in.

The term “chimeric antigen receptor” (“CAR”, also known as an “artificial T cell receptor”, “chimeric T cell receptor” and “chimeric immunoreceptors”) should be understood as a reference to engineered receptors which graft an antigen recognition moiety onto an immune cell. Generally speaking, a CAR is composed of an antigen recognition moiety specific for a target antigen, a transmembrane domain, and an intracellular/cytoplasmic signaling domain of a receptor natively expressed on an immune cell, operably linked to each other. By “operably linked” is meant that the individual domains are linked to each other such that upon binding of the antigen recognition moiety to target antigen, a signal is induced via the intracellular signaling domain to activate the cell that expresses the CAR (e.g., a T cell or an NK cell) and enable its effector functions to be activated.

The antigen recognition moiety of CARs is an extracellular portion of the receptor which recognizes and binds to an epitope of a target antigen. The antigen recognition moiety is usually, but not limited to, an scFv.

The intracellular domain of a CAR can include a primary cytoplasmic signaling sequence of a naturally occurring receptor of an immune cell, and/or a secondary or costimulatory sequence of a naturally occurring receptor of an immune cell. Examples of primary cytoplasmic signaling sequences include those derived from TCR zeta, FcR gamma, FcR beta, CD3 gamma, CD3 delta, CD3 epsilon, CD5, CD22, CD79a, CD79b, and CD66d. In some embodiments, the intracellular signaling domain of a CAR comprises a cytoplasmic signaling sequence from CD3-zeta. In some embodiments, the intracellular signaling domain of a CAR can comprise a cytoplasmic signaling sequence from CD3-zeta in combination with a costimulatory signaling sequence of a costimulatory molecule. Examples of suitable costimulatory molecules include CD27, CD28, 4-4BB (CD137), OX40, CD30, CD40, PD-1, TIM3, ICOS, lymphocyte function-associated antigen-1 (LFA-1), CD2, CD7, LIGHT, NKG2C, B7-H3, and the like. In some embodiments, the cytoplasmic domain of a CAR is designed to comprise the signaling domain of CD3-zeta and the signaling domain of CD28.

The transmembrane domain of a CAR is generally a typical hydrophobic alpha helix that spans the membrane and may be derived from any membrane-bound or transmembrane protein. The transmembrane domain may be derived either from a natural or from a synthetic source. Where the source is natural, the domain may be derived from any membrane-bound or transmembrane protein. For example, transmembrane regions may be derived from the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively, the transmembrane domain may be synthetic, in which case it will comprise predominantly hydrophobic residues such as leucine and valine.

The terms “target antigen” should be understood as a reference to any proteinaceous or non-proteinaceous molecule expressed by a cell which is sought to be targeted by the receptor-expressing immune cells such as T cells or NK cells. A target antigen may be a “self” molecule (a molecule expressed in the body of a patient) or a non-self molecule (e.g., from an infectious microorganism). Target antigens referred to herein are not limited to molecules which are naturally able to elicit a T or B cell immune response; rather, a “target antigen” is a reference to any proteinaceous or non-proteinaceous molecule which is sought to be targeted. In some embodiments, a target antigen is expressed on the cell surface. It should be understood that a target antigen may be exclusively expressed by the target cell, or it may also be expressed by non-target cells. In some embodiments, a target antigen is a non-self molecule, or a molecule that is expressed exclusively by the cells sought to be targeted or expressed by the cells sought to be targeted at a significantly higher level than by normal cells. Non-limiting examples of target antigens include the following: differentiation antigens such as MART-1/MelanA (MART-I), gplOO (Pmel 17), tyrosinase, TRP-1, TRP-2 and tumor-specific multilineage antigens such as, MAGE-1, MAGE-3, BAGE, GAGE-1, GAGE-2, p15; overexpressed glycoproteins such as MUC1 and MUC16; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other tumor associated antigen include folate receptor alpha (FRα), EGFR, CD47, CD24, TSP-180, MAGE-4, MAGE-5, MAGE-6, RAGE, NY-ESO, pl 85erbB2, pl80erbB-3, cMet, nm-23HI, PSA, CA 19-9, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27. 29\BCAA, CA 195, CA 242, CA-50, CAM43, CD68\P1, CO-029, FGF-5, G250, Ga733\EpCAM, HTgp-175, M344, MA-50, MG7-Ag, NB/70K, NY-CO-1, RCAS 1, SDCCAG16, TA-90\Mac-2 binding protein\cyclophilin C-associated protein, TAAL6, TAG-72, TLP, TPS, PSMA, mesothelin, or BCMA.

In some embodiments, the target antigen is a tumor-associated antigen, in particular a protein, glycoprotein or a non-protein tumor-associated antigen.

In some embodiments, the target antigen is selected from the group consisting of CD47, folate receptor alpha (FRα) and BCMA.

In some embodiments, the target antigen is a tumor-associated antigen, for example, the tumor-associated antigen TAG-72.

In other embodiments, the target antigen is a surface protein, for example CD24, and in another embodiment, a surface protein that can be used for tumor-targeting, for example, CD19 or CD20.

### Pharmaceutical Composition and Therapeutic Use of the Modified Cells

In a further aspect, provided herein are compositions containing the cells produced by the methods disclosed herein, i.e., modified cells in which the function of one or more of the selected genes has been inhibited.

In some embodiments, provided herein is a pharmaceutical composition containing cells produced herein, and a pharmaceutically acceptable carrier. A pharmaceutically acceptable carrier includes solvents, dispersion media, isotonic agents and the like. Examples of carriers include oils, water, saline solutions, gel, lipids, liposomes, resins, porous matrices, preservatives and the like, or combinations thereof. In some embodiments, the pharmaceutical composition is prepared and formulated for administration to patients, such as for adoptive cell therapy, typically in a unit dosage injectable form (solution, suspension, emulsion). In some embodiments, a pharmaceutical composition can employ time-released, delayed release, and sustained release delivery systems.

In some embodiments, a pharmaceutical composition comprises cells in an amount effective to treat or prevent a disease or condition, such as a therapeutically effective or prophylactically effective amount. In some embodiments, a pharmaceutical composition includes modified cells disclosed herein, in an amount of about 1 million to about 100 billion cells, for example, at least 1, 5, 10, 25, 50, 100, 200, 300, 400 or 500 million cells, up to about 1, 5, 10, 20, 30, 40, 50, 60, 70, 80, 90 or 100 billion cells.

In some embodiments, a pharmaceutical composition further comprises another active agent or drug, such as a chemotherapeutic agent.

In another aspect, provided herein are methods and uses of the modified cells disclosed herein, such as therapeutic methods and uses in adoptive cell therapy.

In some embodiments, a method includes administration of the modified cells disclosed herein or a composition comprising the modified cells disclosed herein to a subject having a disease or condition or at risk of developing the disease or condition.

In some embodiments, the disease or condition is a neoplastic condition (i.e., cancer), a microorganism or parasite infection (such as HIV, STD, HCV, HBV, CMV, COVID-19 or antibiotic resistant bacteria), an autoimmune disease (e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma), fibrosis of an organ (e.g., heart, lung, liver, etc.), or endometriosis.

In some embodiments, a neoplastic condition includes central nervous system tumors, retinoblastoma, neuroblastoma, paediatric tumors, head and neck cancers (e.g., squamous cell cancers), breast and prostate cancers, lung cancer (both small and non-small cell lung cancer), kidney cancers (e.g., renal cell adenocarcinoma), esophagogastric cancers, hepatocellular carcinoma, pancreaticobiliary neoplasias (e.g., adenocarcinomas and islet cell tumors), colorectal cancer, cervical and anal cancers, uterine and other reproductive tract cancers, urinary tract cancers (e.g., of ureter and bladder), germ cell tumors (e.g., testicular germ cell tumors or ovarian germ cell tumors), ovarian cancer (e.g., ovarian epithelial cancers), carcinomas of unknown primary, human immunodeficiency associated malignancies (e.g., Kaposi's sarcoma), lymphomas, leukemias, malignant melanomas, sarcomas, endocrine tumors (e.g., of thyroid gland), mesothelioma and other pleural or peritoneal tumors, neuroendocrine tumors and carcinoid tumors.

In some embodiments, the present method leads to treatment of the condition, i.e., a reduction or amelioration of the condition, or any one or more symptoms of the condition, e.g., by inhibiting tumor growth and/or metastasis in the context of treating a cancer, or by reducing the viral load and/or spread in the context of treating a viral infection. The term “treatment” does not necessarily imply a total recovery. In some embodiments, the present method leads to prophylaxis of a condition, i.e., preventing, reducing the risk of developing, or delaying the onset of the condition. Similarly, “prophylaxis” does not necessarily mean that a subject will not eventually contract the condition.

In some embodiments, the subject, e.g., patient, to whom the cells or compositions are administered is a mammal, typically a primate, such as a human.

In some embodiments, the cells or a composition comprising the cells are administered parenterally. The term “parenteral,” as used herein, includes intravenous, intramuscular, subcutaneous, and intraperitoneal administration.

The desired dosage of modified cells or a composition comprising modified cells can be delivered by a single administration, by multiple administrations, or by continuous infusion administration of the composition. Therapeutic or prophylactic efficacy can be monitored by periodic assessment of a treated subject.

In some embodiments, adoptive cell therapy is carried out by autologous transfer. Immune cells (such as T cell) are isolated and/or otherwise prepared from a subject who is to receive the cell therapy, or from a sample derived from such a subject. In some embodiments, immune cells (e.g., T cells or NK cells) are isolated from a subject, modified in accordance with the methods disclosed herein (to inhibit the function of one or more genes), and then administered to the same subject.

In some embodiments, adoptive cell therapy is carried out by allogeneic transfer, in which the cells are isolated and/or otherwise prepared from a donor subject different from a subject who is to receive the cell therapy (recipient subject). In some embodiments, the donor and recipient subjects express the same HLA class or supertype.

### EXAMPLES

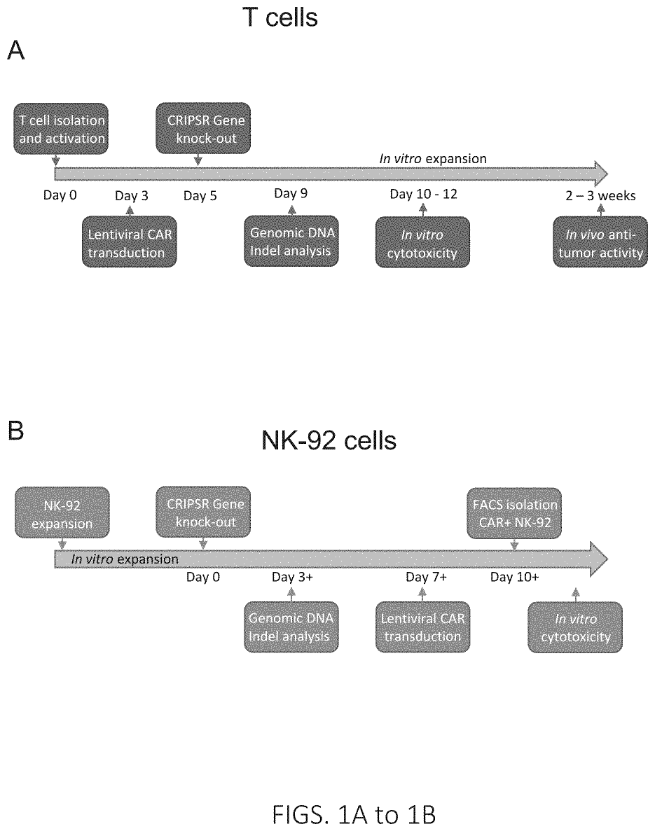

In the following examples, it has been illustrated, without limitation, that to enhance the function of CAR-T cells, T cells, NK cells and derived cells (e.g. iNK cells) for tumor treatment, CRISPR/Cas9 gene editing technology was employed to eliminate the negative immune-regulators of these immune cells. In the case of T cells containing a CAR, the cells were firstly transduced by lentiviral CAR vectors after activation, then a Cas9 nuclease complex with specifically designed a guide RNA was transfected into CAR-T cells to ablate an immune regulator gene(s) (FIG. 1A). In the case of NK-92 cells containing a CAR, the cells were first transfected with a Cas9 nuclease complex with specifically designed guide RNA to ablate an immune regulator gene(s) and then transduced using lentiviral CAR vectors (FIG. 1B). Gene editing efficiency was examined by genomic DNA sequencing-based quantification. The cytotoxicity and expansion rate were then monitored during the in vitro expansion of the cells. To evaluate the in vivo persistence, CAR-cells (in the following examples CAR-T cells) were adoptively transferred into mouse xenograft tumor model (FIGS. 1A-1B).

### Example 1—Generation of Second-Generation TAG-72 CAR-T Cells

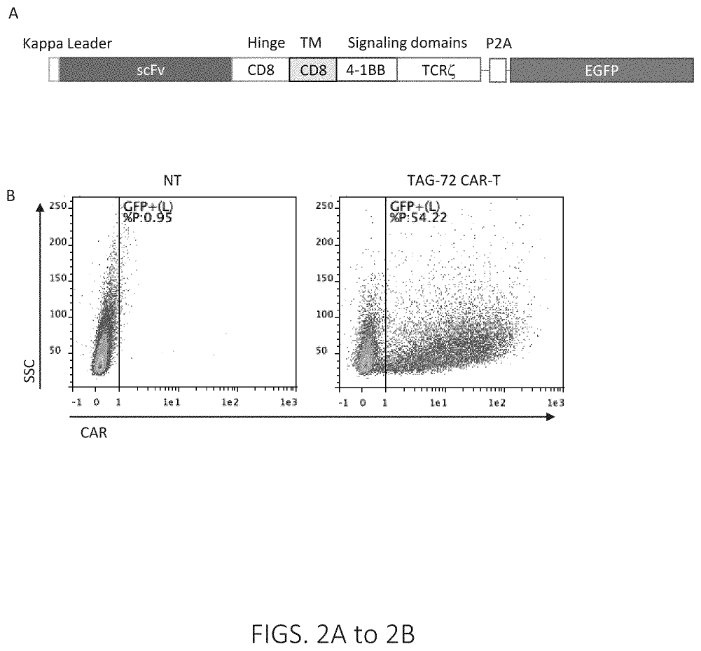

TAG-72 is an established tumor marker for adenocarcinomas and also a target for CAR-T cells in certain solid tumors. Second generation TAG-72 CAR-T cells were generated as described in WO2017/088012, incorporated herein by reference. The TAG-72 CAR expression cassette contained a kappa leader sequence as the signal peptide, an anti-TAG-72 scFv as the tumor antigen binding moiety, the hinge and transmembrane regions from human CD8, and the cytoplasmic activation signaling domains of 4-1BB and CD3 zeta. The P2A is a signal sequence directing proteolytic cleavage, which releases EGFP as a fluorescent reporter of CAR expression (FIG. 2A). Thus, CAR transduction efficiency and expression level in T cells could be detected using GFP flow cytometry after lentiviral transduction (FIG. 2B).

### Human T Cell Isolation and Culture

Primary human T cells were isolated from healthy human donors either from fresh whole blood, or from buffy coats obtained from the Australian Red Cross Blood Service (non-conforming/discarded material not suitable for clinical purposes). All patients and healthy donors provided informed consent. Peripheral blood mononuclear cells (PBMCs) were isolated by Ficoll-Paque (GE Healthcare, Illinois, United States) centrifugation using Leucosep™ tubes (Greiner, Kremsmunster, Austria) as per manufacturer's instructions. PBMCs were cryopreserved prior to use. For use in transductions and transfections, PBMCs were thawed and T cells were isolated and activated using Dynabeads® Human T-Activator CD3/CD28 beads (Thermofisher, Massachusetts, United States). Cells and beads were incubated at 1:3 ratio for 1 hour at room temperature, with continual gentle mixing. Unbound cells were then removed by placing cell-bead suspension on a magnet for 1-2 mins. The supernatant was removed and cell-bead mixture was incubated for ˜65 hrs at 37° C. 5% CO₂ in T cell medium: TexMACS Medium (Miltenyi Biotech, Bergisch Gladbach, Germany) with 5% human AB serum (Sigma-Aldrich, Missouri. United States) and 100 U/mL IL-2. T cells were collected by dissociation of the cell-bead complexes by mixing 20-50×, immediately placed on a magnet for 1-2 mins and the cell containing supernatant collected. The isolated T cell suspension was counted on a MUSE™ cell counter (Merck-Millipore, Massachusetts, United States) and prepared for transfection.

### Lentiviral Transduction

Lentiviral CAR vectors were used to transduce the activated human CD3+ T cells as described in WO2017/088012, incorporated herein by reference. To produce the lentiviral CAR-T cells, the activated human CD3+/CD28+ T cells were incubated with the lentiviral particles in RetroNectin® (Takara Bio Inc) coated plates for 48 hours.

### Flow Cytometry of CAR Expression.

To detect the expression of CAR construct in lentiviral transduced CAR-T cells, flow cytometric analysis was performed on a MACSQuant® Analyzer 10 (Miltenyi Biotec, Bergisch Gladbach, Germany). GFP expression was analysed. Propidium iodide solution (Miltenyi Biotec) or Viobility 405/520 dye was used to discriminate live and dead cells.

### Example 2—Generation of Gene Edited TAG-72 CAR-T Cells Using CRISPR

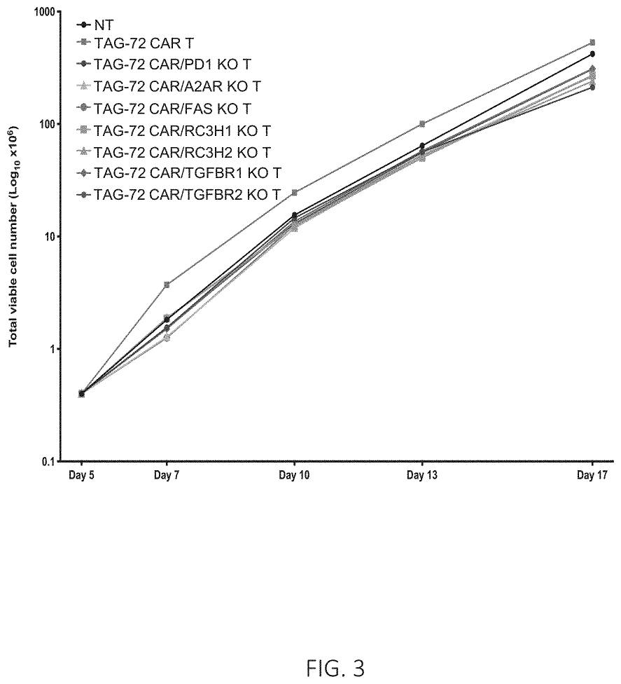

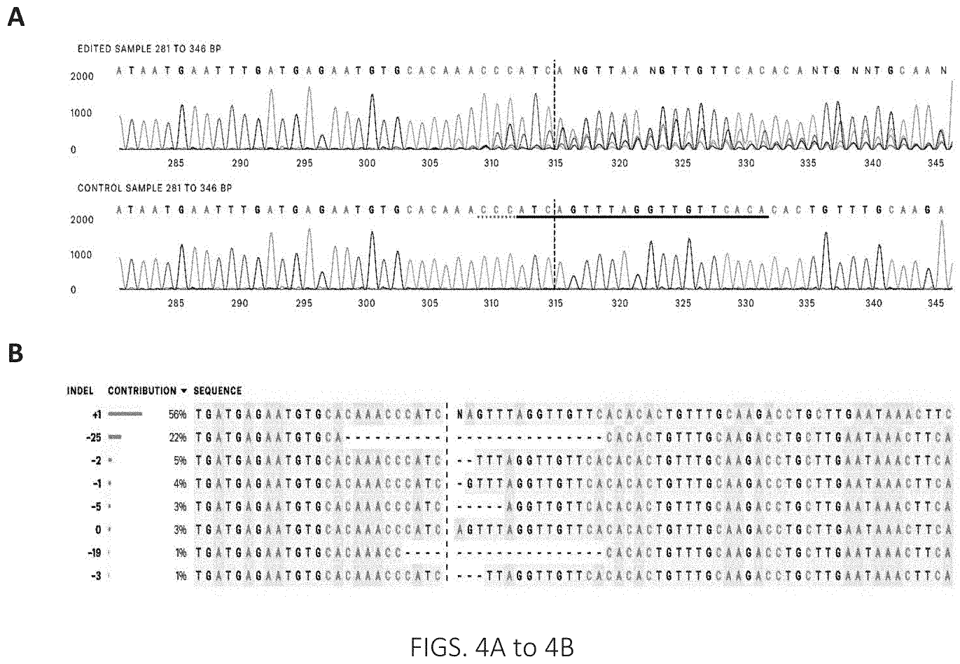

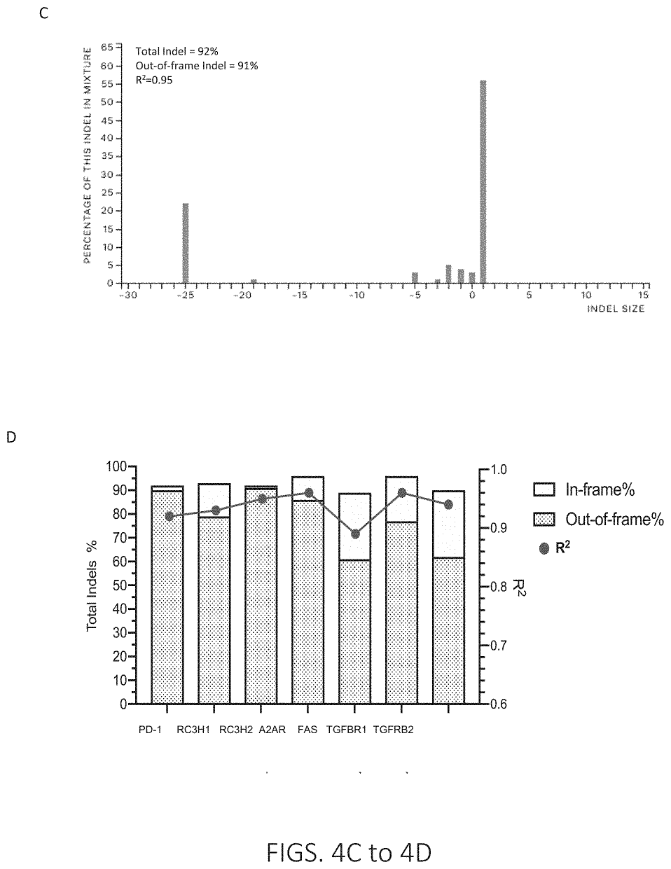

To generate CRISPR gene knock-out (KO) CAR-T cells, RNP complex formed by representative guide RNAs (PD1 KO, SEQ ID NO: 1; RC3H1 KO, SEQ ID NO: 2: RC3H2 KO, SEQ ID NO: 4; A2AR KO, SEQ ID NO: 7; FAS KO, SEQ ID NO: 9; TGBFBR1 KO, SEQ ID NO: 11; TGFBR2 KO, SEQ ID NO: 14) were transfected into T cells 48 hours after lentiviral TAG-72 CAR transduction at Day 5, respectively (FIGS. 1A to 3). Though electroporation induced cell death occurred, the RNP transfected CAR-T cells could be recovered and expanded as well as the non-transfected CAR-T cells using the protocol disclosed herein (FIG. 3). Four days after RNP transfection, the genomic DNA of CAR-T cells was extracted for quantitative analysis of gene editing. The gene editing efficiency was analysed using the ICE (Inference of CRISPR Edits) assay (Hsiau et al., Inference of CRISPR Edits from Sanger Trace Data. bioRxiv, 2018, 10.1101/251082 (251082). RC3H2 gene editing efficiency analysis was shown here as a representative result of ICE assay (FIGS. 4A to 4C). RC3H2 gRNA (SEQ ID NO: 4) showed high activity to introduce indels (total indel frequency=92%) into the early exon of RC3H2 gene. In addition, it resulted in high frequency of frameshift (out-of-frame indel frequency=91%) of the open reading frame, thereby disrupting the translation of functional RC3H2 protein (FIGS. 4B and 4C). For all the CRISPR gene edited CAR-T cells in the present study, very high gene editing efficiencies (total indel percentage=89% to 96%) and efficient gene knock-out outcomes (out-of-frame indel frequency=61% to 91%) were achieved (FIG. 4D). In summary, these results indicate that the gRNAs used in the study are verified to have high activity to disrupt the expression of a corresponding gene in CAR-T cells without perturbation of in vitro expansion of the CAR-T cells.

### CRISPR Gene Editing of CAR-T Cells

Two days after lentiviral TAG-72 CAR transduction, T cells were washed by dPBS for Cas9 RNP transfections. crRNAs and tracrRNA (Synthego or IDT) were annealed to form the full-length guide RNAs. Cas9 RNPs were prepared before transfection by incubating Cas9 protein with the full-length gRNAs at 1:2 ratio at room temperature for 10 to 20 minutes. To transfect the Cas9 RNP, T cells were electroporated with a Neon transfection device (Thermofisher) or 4D-Nucleofector device (Lonza, Basel, Switzerland).

### Quantitative Assessment of Genome Editing

The efficacy and the mutation spectrum of CRISPR/Cas9 genome editing efficiency were analysed by ICE assay (Hsiau et al., Inference of CRISPR Edits/from Sanger Trace Data. bioRxiv, 2018, 10.1101/251082(251082)). Genomic DNA was extracted from cells 4 days after electroporation using ISOLATE II Genomic DNA Kit (Bioline) following manufacturer's instructions. PCR amplicons spanning the gRNA genomic target sites were generated using the High-Fidelity Taq polymerase (New England Biolabs). The purified PCR products were Sanger-sequenced and the sequence chromatogram was analysed with the ICE software available on line.

### Example 3—In Vitro Function of CRISPR Gene Edited TAG-72 CAR-T Cells

During the expansion phase of gene edited TAG-72 CAR-T cells, the tumor killing ability of the cells were evaluated using xCELLigence® real-time assay in vitro before in vivo assessment. Gene edited TAG-72 CAR-T cells were generated and verified as describe in Examples 1 and 2.

### T Cell In Vitro Cytotoxicity Assay

The real-time cell monitoring system (xCELLigence®) was employed to determine the killing efficiency of CAR-T cells in vitro. 10,000 target cells/100 μL (for example, the ovarian cancer cell line OVCAR-3) were resuspended in culture media (for example, RPMI-1640 basal media) supplemented with 10%-20% fetal calf serum and bovine insulin and deposited into RTCA plates. Target cells were maintained at 37° C., 5% CO₂ for 3-20 h to allow for cellular attachment. Following attachment of target cells, TAG-72 CAR-T effector cells were added at various effector to target ratios ranging from 1:5 to 5:1. In some instances, effector cells were isolated based on GFP expression via FACS prior to use. In parallel, non-transfected T cells were co-cultured with target cells to demonstrate the background functionality of T cells in vitro. All co-cultures were maintained in optimal growth conditions for at least 20 h. Throughout, cellular impedance was monitored; a decrease in impedance is indicative of cell detachment and ultimately cell death.

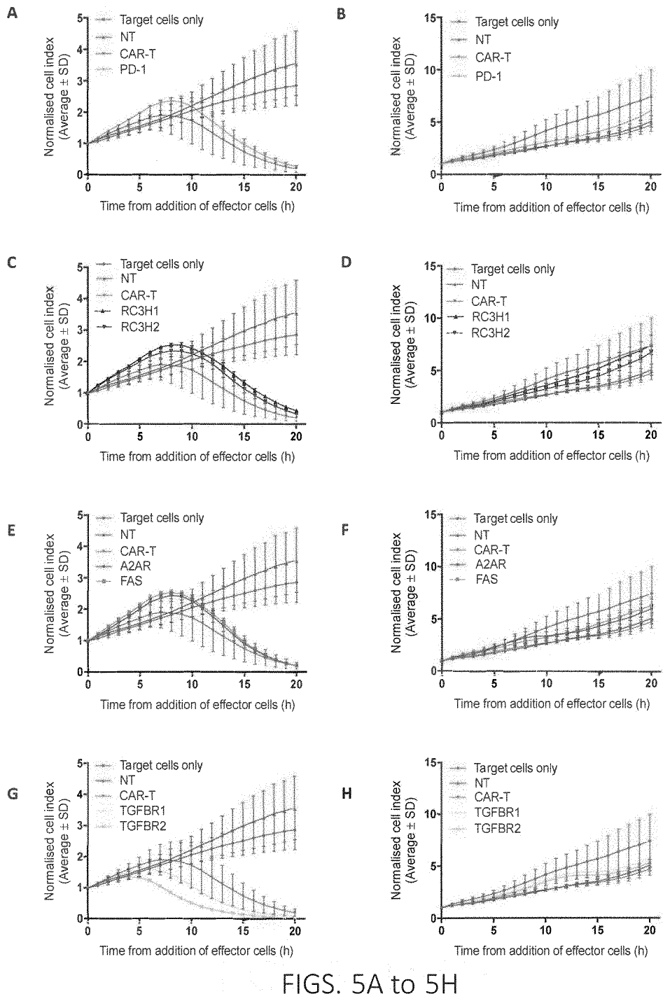

To compare the initial capacity of the gene edited lentiviral TAG-72 CAR-T cells to lyse tumor cells, tumor cells with high or low TAG-72 expression were incubated with gene edited CAR-T cells or other negative control effector T cells (non-transfected), and in vitro cytotoxicity was monitored by xCELLigence®. All these gene edited TAG-72 CAR-T cells killed TAG-72 high tumor cells (OVCAR-3) as efficiently as TAG-72 CAR-T cells (FIGS. 5A, C, E and G), whereas no lysis of TAG-72 low tumor cells (MES-OV) was observed (FIGS. 5B, D, F and H). These results indicate that the gene edited TAG-72 CAR-T cells generated using the CRISPR procedure disclosed herein retain the tumor killing capacity and specificity of TAG-72 CAR-T cells.

### Example 4—In Vivo Function of CRISPR Gene Edited TAG-72 CAR-T Cells

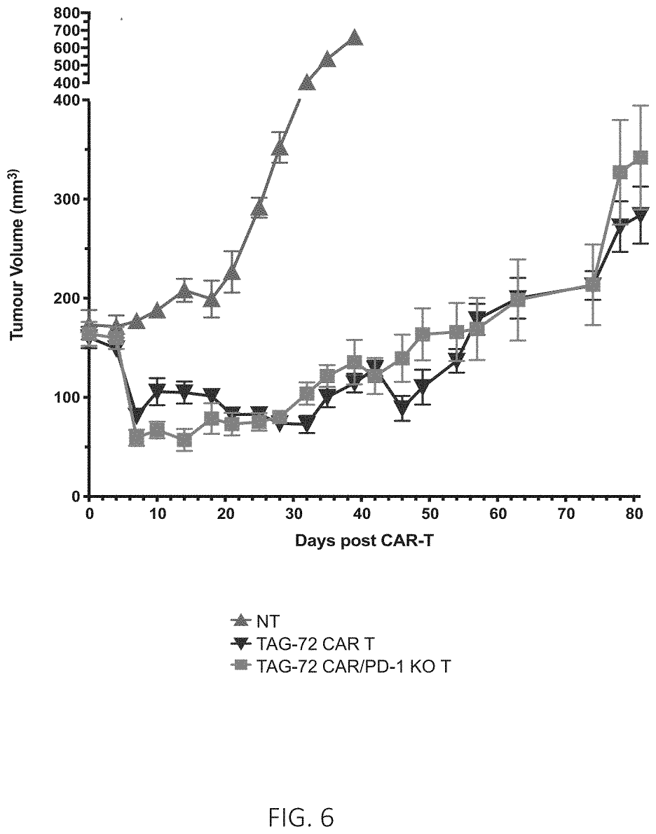

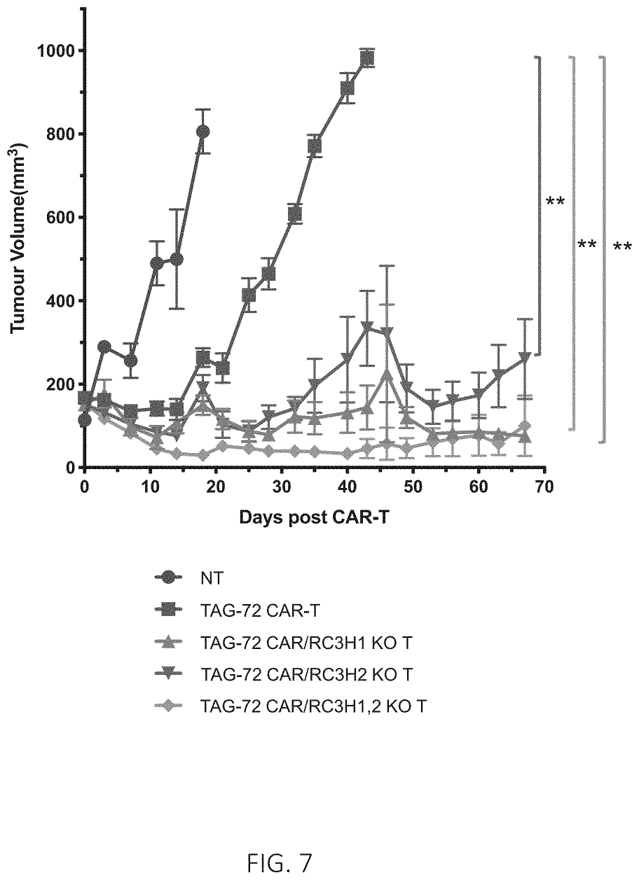

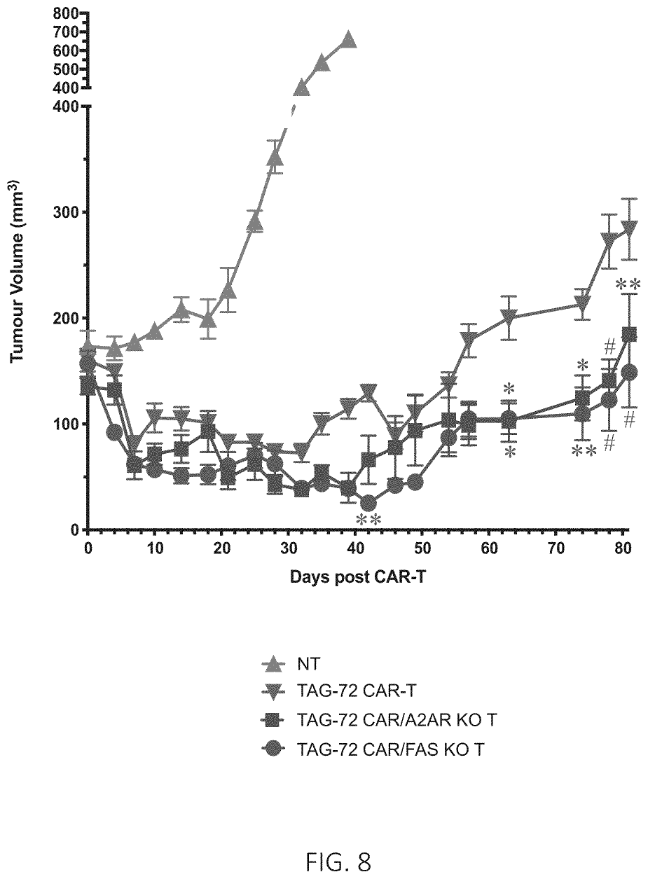

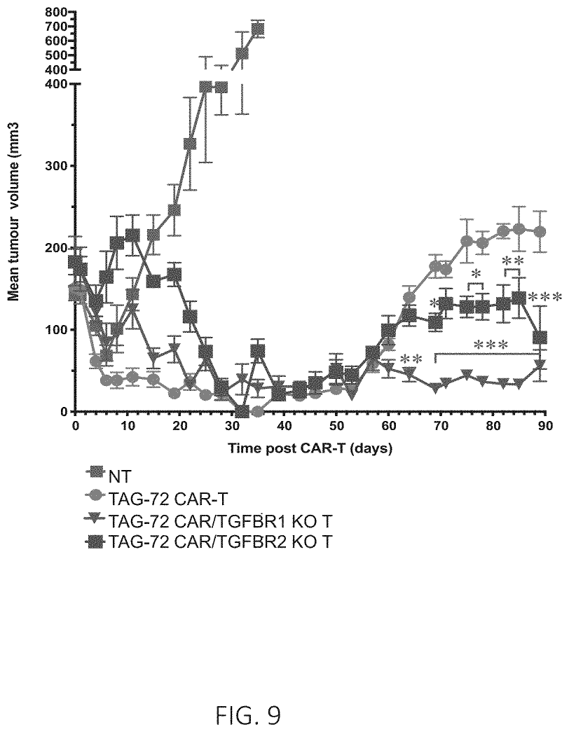

A recent study showed that TAG-72 CAR-T cells could reduce the in vivo ovarian tumor burden but could not persist to prevent tumor recurrence (Murad, J. P., et al., Effective Targeting of TAG72(+) Peritoneal Ovarian Tumors via Regional Delivery of CAR-Engineered T Cells. Front Immunol, 2018, 9: p. 2268). TAG-72 CAR-T cells, which were generated and verified as described in Example 1, 2 and 3, were assessed for their efficacy in an in vivo mouse solid tumor (xenograft) model. For this model, human tumor cell lines were grown on the flank of NSG mice by subcutaneously injecting approximately 1×10⁷ human-derived TAG-72 positive OVCAR-3 cancer cells into the flanks of 6 to 10-week-old mice. Within 7 to 9 weeks, fully formed 150-200 mm³ tumors developed at the injection site. Once tumors reached this volume, the groups were randomized for treatment. CAR-T cells with different edited genes were administered to the mice intravenously, with a total of 2 injections of 5×10⁶ T cells per injection. The tumor volume, body weight and clinical score were monitored after CAR-T cell infusion. Mice with tumor size from 800 mm³ to 1000 mm³, significant weight loss or poor clinical score were culled, according to animal ethics approvals. In this ovarian cancer tumor model, second generation TAG-72 CAR-T cell treatment reduced the size of tumors initially, but tumor recurrence was observed at around 30 days post CAR-T cell administration (FIG. 6). Gene edited TAG-72 CAR-T cells were generated according to the methods described in Examples 1 and 2 and assessed for in vivo efficacy in the same model. The PD-1 gene knock-out TAG-72 CAR-T cells did not improve the anti-tumor activity or persistence of the TAG-72 CAR-T cells (FIG. 6). However, knock-out of the RC3H1 and/or RC3H2 genes resulted in significantly improved anti-tumor activity and persistence of TAG-72 CAR-T therapy. Moreover, the RC3H1 and RC3H2 double gene knock-out TAG-72 CAR-T cells (TAG-72 CAR/RC3H1,2 KO T cells) showed the best anti-tumor activity and persistence in these groups, as was evidenced by complete prevention of tumor recurrence in the TAG-72 CAR/RC3H1,2 KO T cells treated mice over the monitoring period (FIG. 7). A2AR and FAS gene knock-out also improved the anti-tumor efficacy and durability of TAG-72 CAR-T therapy, which delayed the recurrence of tumor in the NSG mice xenograft model (FIG. 8). Dominant negative mutation of TGFβ receptor 1 and 2 directed by CRISPR also enhanced the persistence of TAG-72 CAR-T cells, evidenced by more durable control of tumor volume from 60 days after CAR-T treatment (FIG. 9).

### Example 5—Generation of RC3H1 AND/OR RC3H2 Gene Edited CD19 CAR-T Cells Using CRISPR and In Vivo Function

CD19 CAR-T cell therapy is the first successful CAR-T treatment approved for B cell malignancies (Porter et al., N Engl J Med, 2011. 365(8): p. 725-33). In order to verify that the anti-tumor activity of CAR-T cells enhanced by CRISPR gene knock-out is not limited to OVCAR-3 tumor model, TAG-72 antigen or TAG-72 CAR-T cells, CD19 CAR-T cells with RC3H1 and/or RC3H2 gene knock-out were also generated for functional evaluation in vivo. The CD19 scFv-4-1BB-CD3ζ CAR expression cassette was constructed as described previously (Porter et al., N Engl J Med, 2011. 365(8): p. 725-33; Milone et al., Mol Ther, 2009. 17(8): p. 1453-64; see, also, WO2017088012). CD19 scFv-4-1BB-CD3ζ CAR lentiviral vectors were produced and transduced into human activated T cells to generate the CD19 CAR-T cells as described in Example 1, and then transfected by RNP complex formed by RC3H1 gRNA (SEQ ID NO: 2) and/or RC3H2 gRNA (SEQ ID NO: 4) as described in Example 2, to generate the CRISPR RC3H1 and/or RC3H 2 gene knock-out CD19 CAR-T cells.

### CD19 CAR-T Cells In Vivo Cytotoxicity Assay

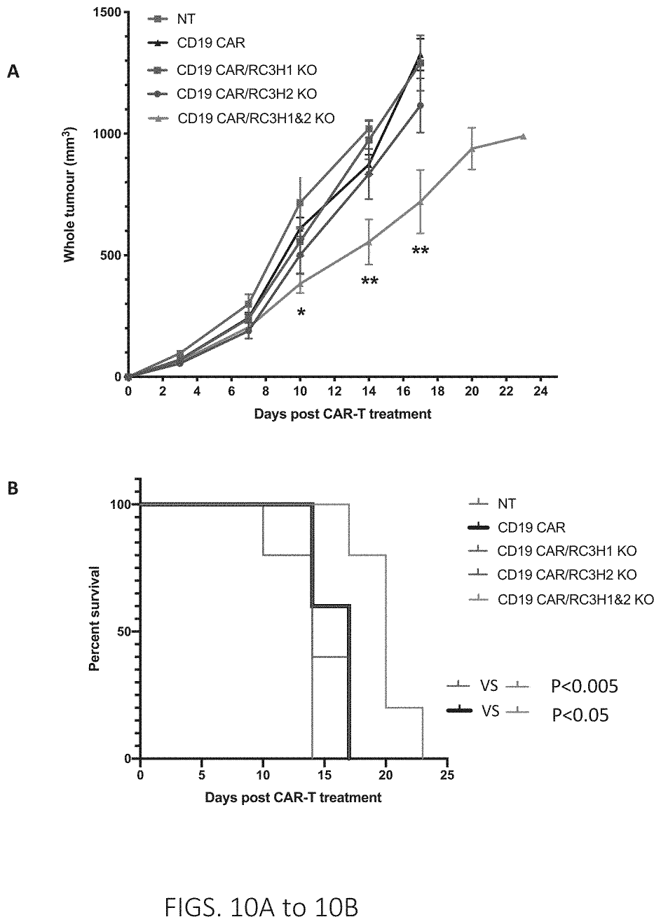

The in vivo efficacy of T cells was assessed in a Burkitt's lymphoma xenograft model. For this model, 5×10⁵ CD19 positive Raji lymphoma cells were injected subcutaneously into the flanks of 6 to 10-week-old NSG mice. A single dose of 5×10⁶ CAR-T cells was injected intravenously per mouse 3 days after tumor inoculation. The tumor volume, body weight and clinical score were monitored after CD19 CAR-T cell infusion. Mice with a tumor size from 800 mm³ to 1000 mm³, significant weight loss or poor clinical score were culled, according to animal ethics approvals. In this lymphoma tumor model, CD19 CAR/RC3H1,2 KO T cell treatment delayed tumor growth in mice significantly and improved the median survival of tumor bearing mice as compared to CD19 CAR-T cell treatment (FIGS. 10A-10B). This result showed that knock-out of the RC3H1 and RC3H2 genes improved the anti-tumor activity of CD19 CAR-T cells in vivo, similar to what had been observed with TAG-72 CAR-T cells.

### Example 6—Activation Markers of CD19 CAR/RC3H1 and/or RC3H2 KO T Cells after Continued Activation Exposure

The RC3H1 and/or RC3H2 gene knock-out CD19 CAR-T cells were generated as described in Example 5.

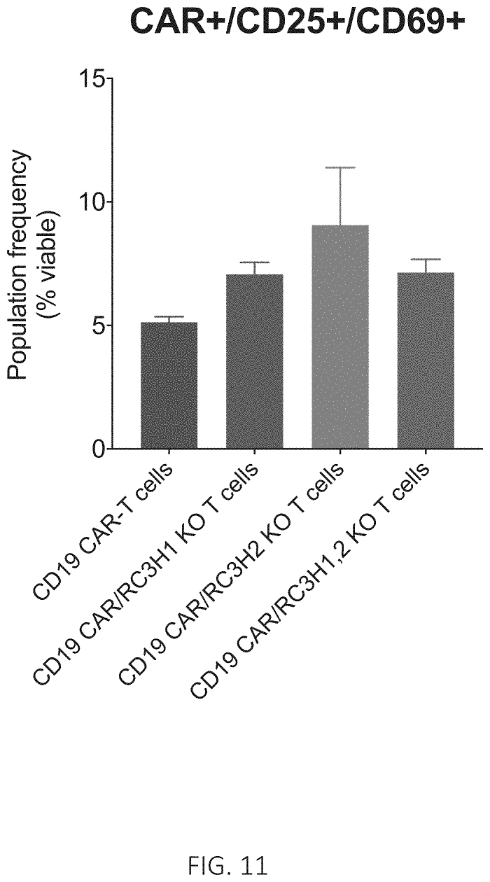

CD19 CART cells±RC3H1 and/or RC3H2 KO were assessed for differences in activation markers following antigen exposure (FIG. 11). The engineered CD19 overexpressing cell line, OVCAR-3(CD19) was irradiated (30 Gy) and seeded at 80,000 cells/mL/well of a 24 well tissue culture plate. Aliquots of 1×10⁶ CAR-T cells (with and without RC3H1 and/or RC3H2 KO) were added to each well at day 0; these CAR-T cells were subsequently transferred daily to an untouched monolayer of irradiated OVCAR-3(CD19) cells over a 7-day period. Following 7 days of continued antigen exposure, effector cells were washed once via centrifugation and assessed for the expression of activation the markers CD69 and CD25. These markers have been associated with early and late activation respectively where expression is linked with TCR ligation. To detect the expression of these activation markers on CAR-T cells, flow cytometric analysis was performed using a MACSQuant® Analyzer 10. CAR expression was detected indirectly by detection of co-expressed GFP. Cell surface staining for CD69 and CD25 was performed using a standard protocol, where cells were incubated with fluorescently conjugated antibodies for 15 min at 4° C., protected from light. Cells were washed twice with FACS buffer before analysis. Propidium iodide solution was used to discriminate live and dead cells. Data analysis was performed using FlowLogic™ software (Miltenyi Biotec).

Following continued antigen exposure, CD19 CAR/RC3H1 and/or RC3H2 KO T cells, lacking either or both genes, showed evidence for a higher frequency of CAR+/CD25+/CD69+ expressing cells. While the increase was not statistically significant, it was consistent across all three KO T cells, indicating increased activation compared to the non-transfected CD19 CAR-T cells (FIG. 11).

### Example 7—Generation of RC3H1 and/or RC3H2 KO T Cells Using CRISPR and In Vitro Function

To demonstrate that the method for generating gene knock-out immune cells is not limited to CAR-T cells, equivalent CRISPR gene knock-out was also performed in normal T cells.

To generate CRISPR T cells, human T cells were isolated and activated using CD3/CD28 beads as described in Example 1. The activated human T cells were transfected by RNP complex formed by RC3H1 gRNA (SEQ ID NO: 2) and/or RC3H2 gRNA (SEQ ID NO: 4) 3 days after activation and expanded in vitro as described in Example 2.

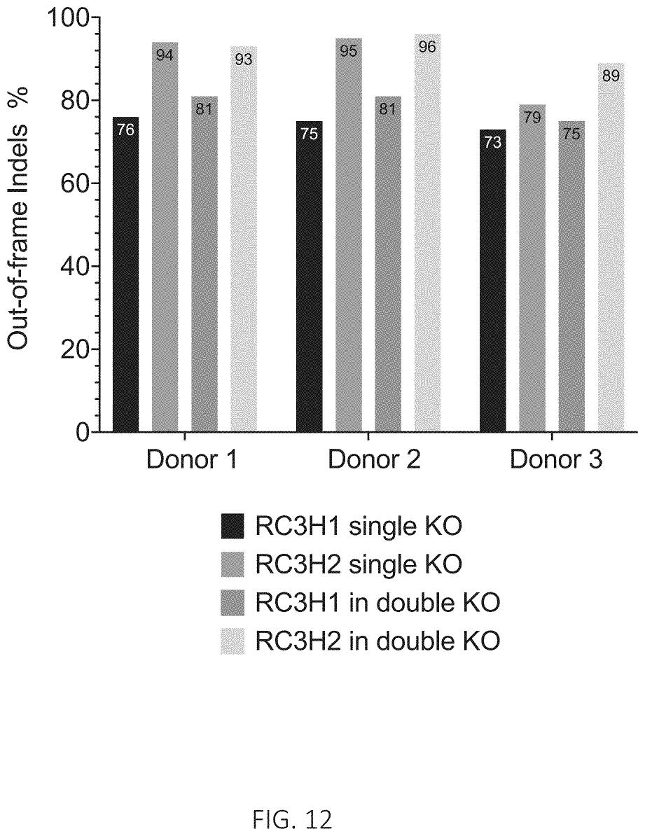

The CRISPR indel frequency and gene knock-out efficiency of the transfected T cells were also analysed by ICE assay as described in Example 2. The ICE assay result showed that these guide RNAs also showed high activity to introduce indels including out-of-frame indels in activated human T cells (FIG. 12).

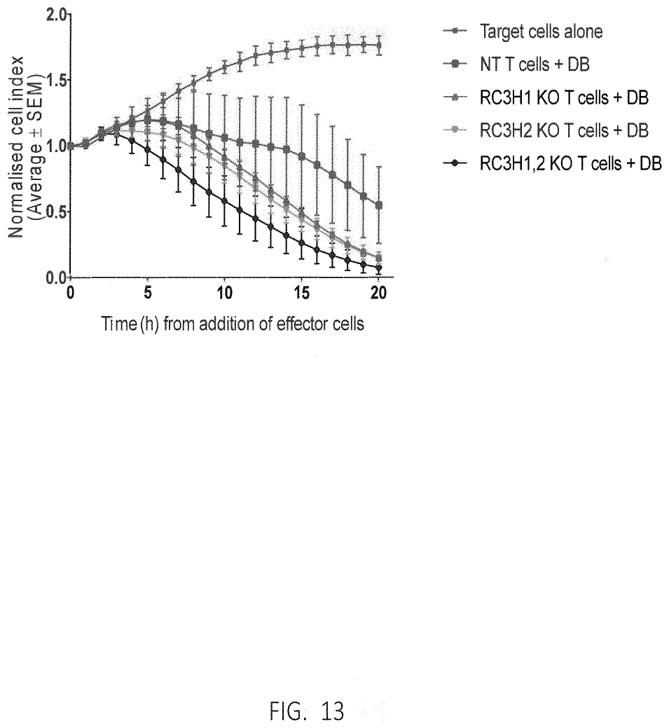

### In Vitro Killing by Prolonged Activation of T Cells (FIG. 13)

To determine whether the effects of the KOs were restricted to CAR-T cells, normal T cells were polyclonally activated for through their TCR and CD28 co-accessory molecules (FIG. 13). RC3H1 and/or RC3H2 KO T cells were maintained in the presence of αCD3/αCD28 beads for at least 92 h at a bead to cell ratio of 1:1. Cell counts were performed approximately every 24 h where fresh beads were added accordingly. Following continued activation, RC3H1 and/or RC3H2 KO T cells displayed improved function in vitro compared to non-transfected (NT) T cells over the 20 h monitoring period. While the differences were not statistically significant, each of the three KO T cells were more efficient in killing target tumor cells than non-transfected T cells, indicating that the prolonged activation of the KO T cells may not result in “exhaustion” of killing function.

### Example 8—Generation of RC3H1 and/or RC3H2 KO NK-92 Cells (with and without CARs) Using CRISPR

To demonstrate that the method for generating gene knock-out immune cells is not limited to just T cells, equivalent CRISPR gene knock-out was also performed in NK-92 cells (FIG. 1B). NK-92 is a Natural Killer (NK) cell line with high cytotoxicity to cancer targets. NK-92 function can be improved through gene modifications including CAR expression (Klingemann et al., Front Immunol, 2016. 7: p. 91). The NK-92 cell line was maintained and expanded in RPMI-1640 medium with 200 U/mL IL-2 and fetal bovine serum.

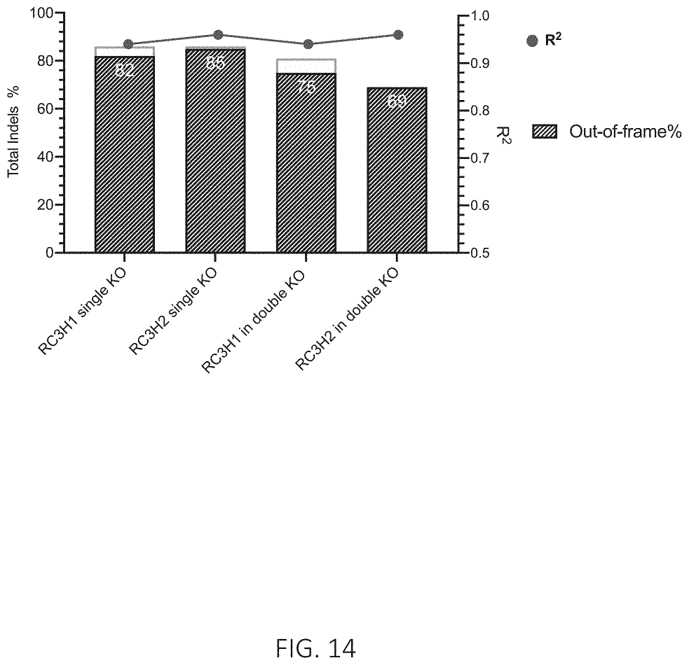

To generate the RC3H1 and RC3H2 gene knock-out NK-92 cells (RC3H1 KO NK-92 cells and RC3H2 KO NK-92 cells, respectively), the NK-92 cells were transfected with RNP complex formed by RC3H1 gRNA (SEQ ID NO: 2) and/or RC3H2 gRNA (SEQ ID NO: 4) as described in Example 2. The CRISPR indel frequency and gene knock-out efficiency of the transfected NK-92 cells were also analysed by ICE assay as described in Example 2. The ICE assay result showed that these guide RNAs could also introduce indels including out-of-frame indels in NK-92 cells at high frequency (FIG. 14).

To generate the TAG-72 CAR/RC3H1 and/or RC3H2 KO NK-92 cells, RC3H1 and/or RC3H2 KO NK-92 cells were transduced using TAG-72 CAR lentiviral vectors as described in Example 1.

### Example 9—In Vitro Function of RC3H1 and/or RC3H2 KO NK-92 and TAG-72 CAR/RC3H KO NK-92 Cells

Lentiviral TAG-72 CAR vectors were used to transduce resultant RC3H1 and/or RC3H2 KO NK-92 cells as described in Example 8. RC3H1 and/or RC3H2 KO NK-92+ CAR cells were generated and routinely maintained in culture in RPMI-1640 with L-glutamine supplemented with 10% FBS and 100 U/mL IL-2. Following at least 3 days in culture, the transduction efficiency was assessed by flow cytometry. Additionally, the ability for RC3H1 and/or RC3H2 KO NK-92±CAR cells to eliminate cancer cells was evaluated in vitro.

The real-time cell monitoring system (xCELLigence®) was employed to determine the killing efficiency of RC3H1 and/or RC3H2 KO NK-92 cells in vitro. Target cells (10,000 target cells per 100 uL) (for example the ovarian cancer cell lines MES-OV or OVCAR-3) were resuspended in culture media (for example, McCoy's 5a or RPMI-1640 basal media) supplemented with 10-20% FBS, with (OVCAR-3) or without (MES-OV) bovine insulin and dispensed into RTCA plates. Target cells were maintained at 37° C., 5% CO₂ for at least 5 hrs to allow for cellular attachment. Following attachment of target cells, RC3H1 and/or RC3H2 KO NK-92 effector cells were added at an E:T ratio of 1:1. In parallel, non-transfected NK-92 cells were co-cultured with target cells to demonstrate the background functionality of NK-92 cells in vitro. All co-cultures were maintained in optimal growth conditions for at least 40 hrs. Cellular impedance was monitored throughout.

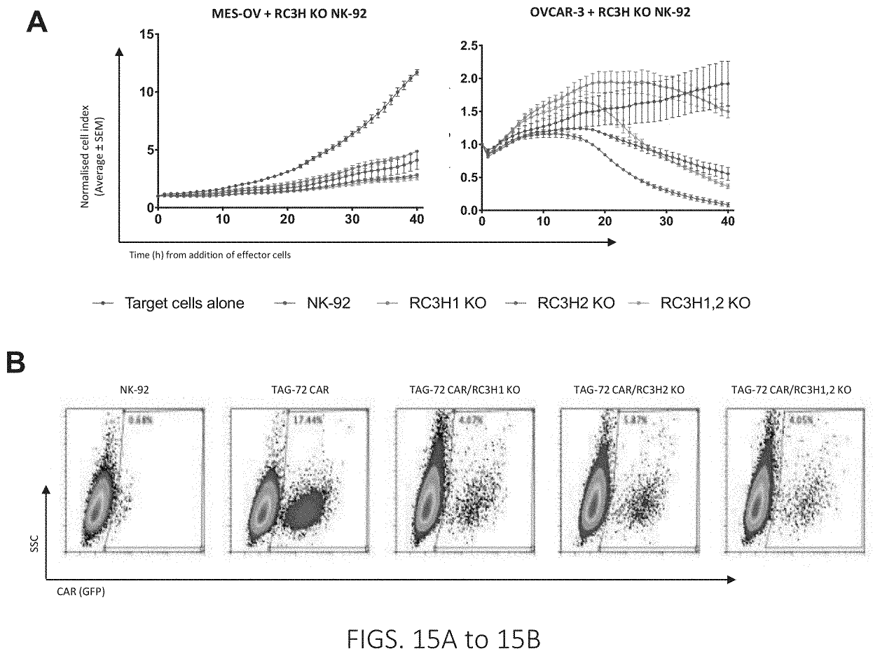

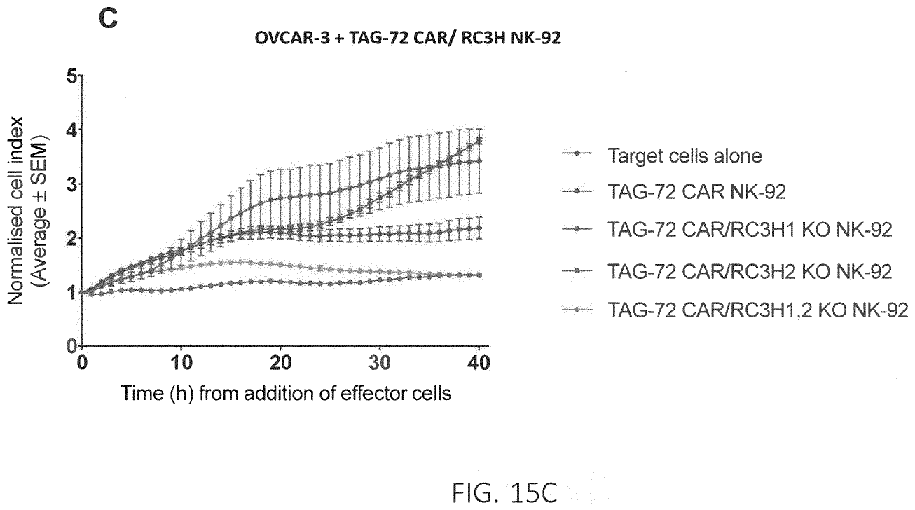

To compare the capacity of RC3H1 and/or RC3H2 KO NK-92 cells to lyse tumor cells, tumor cells were incubated with RC3H1 and/or RC3H2 KO NK-92 cells or non-transfected NK-92 cells and the in vitro cytotoxicity was monitored by xCELLigence®. All NK-92 cells (FIG. 15A, left) demonstrated a cytostatic effect when co-cultured with MES-OV cells. This effect was improved with RC3H2 KO NK-92 cells and RC3H1,2 KO NK-92 cells when compared to the non-transfected NK-92 control, demonstrating an enhancement of function in vitro. Additionally, all NK-92 cells (FIG. 15A, right) demonstrated a cytotoxic effect when co-cultured with OVCAR-3 cells, as demonstrated by a decrease in NCI. This effect was improved with RC2H2 KO NK-92 cells and RC2H1/2 KO NK-92 cells respectively compared to the non-transfected NK-92 control condition demonstrating an enhancement of function in vitro.

A similar assay was performed with TAG-72 CAR NK-92 cells. To confirm the transduction of the TAG-72 CAR NK-92 cells into the RC3H1 and/or RC3H2 KO NK-92 cells, flow analysis was performed (as described in Example 1), where GFP was used as a surrogate for the integration and expression of CAR (FIG. 15B). Values represent the % CAR(GFP) expressed as a percent of viable cells, where debris and doublets were excluded in parental gates.

To compare the capacity of RC3H1 and/or RC3H2 gene knock-out TAG-72 CAR-NK-92 cells to lyse tumour cells, cancer cell lines (in this case OVCAR-3) were co-cultured with TAG-72 CAR NK-92 cells±RC3H1 and/or RC3H2 KOs, and the in vitro cytotoxicity was monitored by xCELLigence®. All NK-92 cells bearing a TAG-72 CAR (FIG. 15C) had a cytotoxic effect when co-cultured with OVCAR-3 cells as demonstrated by a plateau or decrease in NCI. This effect was significantly greater with TAG-72 CAR/RC3H1 KO NK-92 cells, TAG-72 CAR/RC3H2 KO NK-92 cells and TAG-72 CAR/RC3H1,2 KO NK-92 cells within 40 h of co-culture, demonstrating an enhancement of function in vitro.

### Example 10—Generation of Gene KO iPSCs Using CRISPR

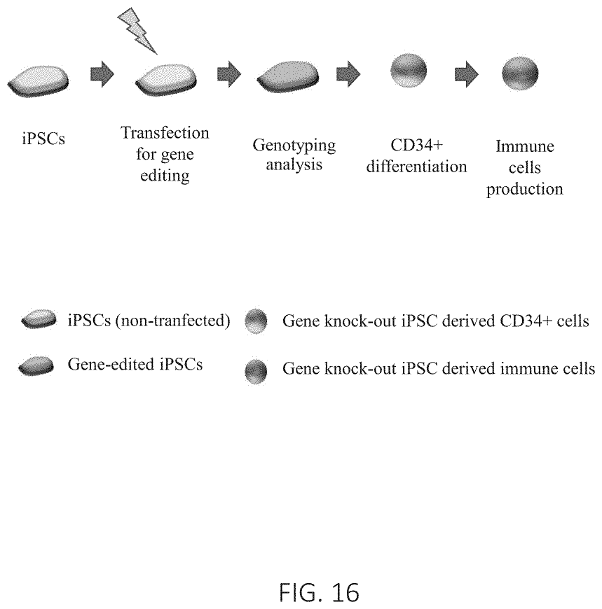

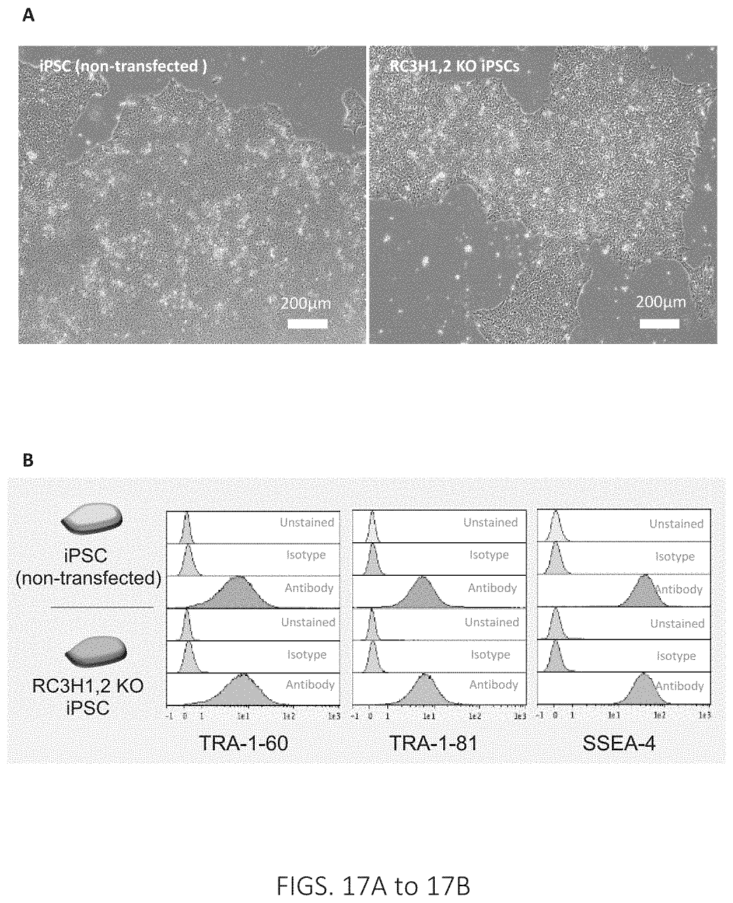

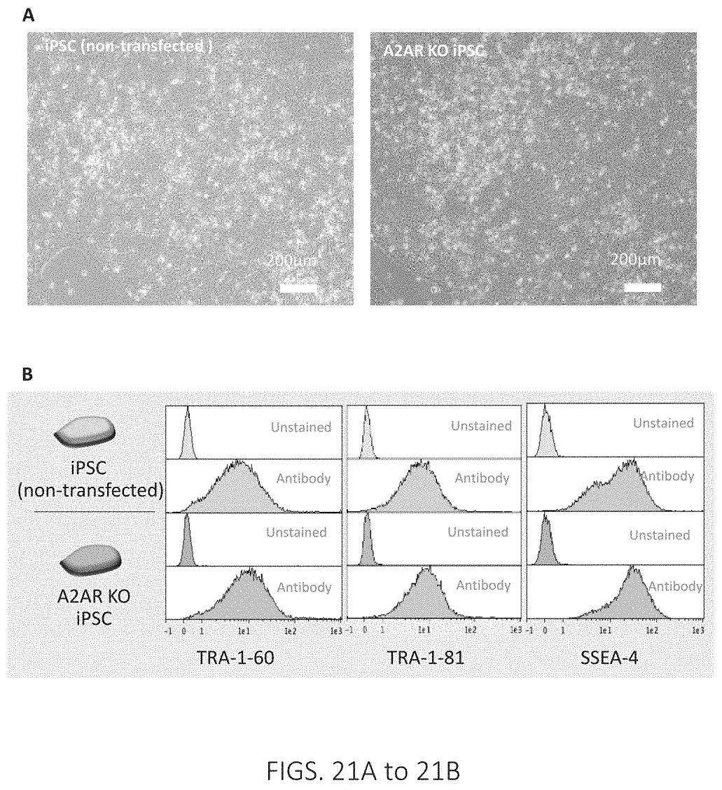

Stem cells such as induced pluripotent stem cells (iPSCs) can self-renew indefinitely and differentiate into various cell types including hematopoietic stem cells (HSCs) and immune cells. Immune cells like T cells and NK cells have previously been generated from iPSCs for cancer therapy (Themeli et al., Nat Biotechnol, 2013. 31(10): p. 928-33; Li et al, Cell Stem Cell, 2018. 23(2): p. 181-192 e5). CRISPR gene knock-out T or NK cells can be derived from iPSCs, following similar methods (FIG. 16). To generate CRISPR RC3H1 and RC3H2 gene double knock-out (RC3H1,2 KO iPSCs) and A2AR gene knock-out iPSCs (A2AR KO iPSCs), RNP complexes formed by representative gRNAs (RC3H1, SEQ ID NO: 2; RC3H2, SEQ ID NO: 4; A2AR, SEQ ID NO: 7) were transfected into iPSCs using the Lonza 4D Nucleofector system. Firstly, a 12 well plate was coated with Laminin-521 (STEMCELL Technologies) in PBS and incubated for 2 hours at 37° C. iPSCs were pre-incubated with mTeSR Plus™ media (STEMCELL Technologies) containing RevitaCell™ Supplement (Life Technologies) for 2 hrs prior to transfection. RNPs were prepared by combining full length gRNAs with Lonza P3 buffer and Cas-9. The RNP mixture was then incubated at room temperature for 10-20 minutes. After pre-incubation, iPSCs were lifted as single cells using Accutas® (Life Technologies) and 1×10⁶ cells per reaction were obtained for electroporation. To generate gene knock-out iPSCs, cells in Lonza P3 buffer and the RNP mixture were combined into PCR tubes, then loaded into the Lonza 4D Nucleofector for electroporation. Following this, mTeSR Plus™ with CloneR™ media (STEMCELL Technologies) were added to the reaction and incubated at room temperature for 10 minutes. After incubation, cells were added to the Laminin-521 pre-coated plate in mTeSR Plus™ with CloneR™ media. Daily media changes with mTeSR Plus™ were performed for 72 hrs and cells were passaged upon reaching ˜80% confluency (6-7 days post-electroporation). After transfection, RC3H1,2 KO iPSCs and A2AR KO iPSCs colonies with pluripotent stem cell-like morphology were maintained in culture (FIGS. 17A and 21A).

Non-transfected and transfected iPSCs were cultured in mTeSR Plus™ on Laminin-521, and imaged at 10× using an EVOS® bright field microscope. The cells were lifted via Accutase® and collected as single cells. They were then stained using antibodies targeting TRA-1-60 (Miltenyi Biotec), TRA-1-81 (STEMCELL Technologies) and SSEA-4 (Miltenyi Biotec), following manufacture recommendations. TRA-1-60, TRA-1-81 and SSEA-4 are surface receptors expressed on pluripotent stem cells and considered common practice to characterise iPSCs (Baghbaderani et al. 2015, Stem Cell Reports). The cells were analysed via MACSQuant® flow cytometer (Miltenyi Biotec), with unstained samples and appropriate isotype controls. Dead cells (via PI staining), debris and doublets were gated out; histogram plots were generated using FlowLogic™ (FIGS. 17A-17B for RC3H1,2 KO and FIGS. 21A-21B for A2AR KO). iPSCs, with or without KO, displayed near identical pluripotency markers for TRA-1-60, TRA-1-81 and SSEA-4, all of which were co-expressed at >95% (FIGS. 17B and 21B). There were no visual differences in iPSCs morphology (FIGS. 17A and 21A) indicating that RC3H1 and RC3H2 double KO or A2AR KO had no negative effect on iPSCs maintenance and pluripotency.

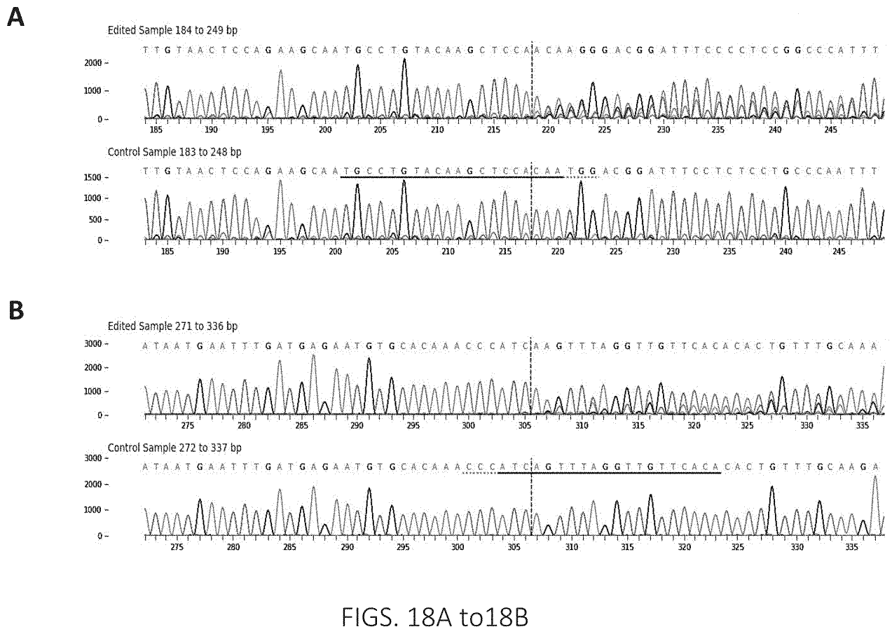

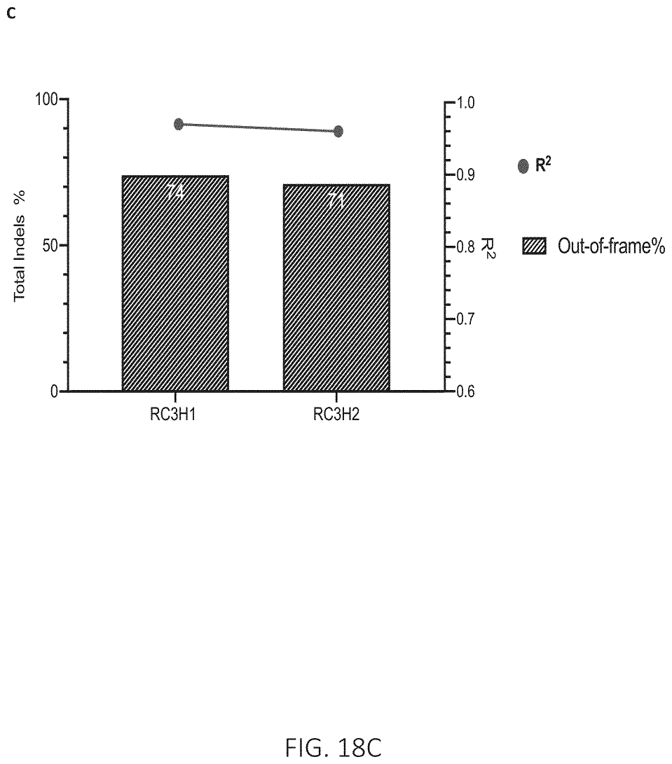

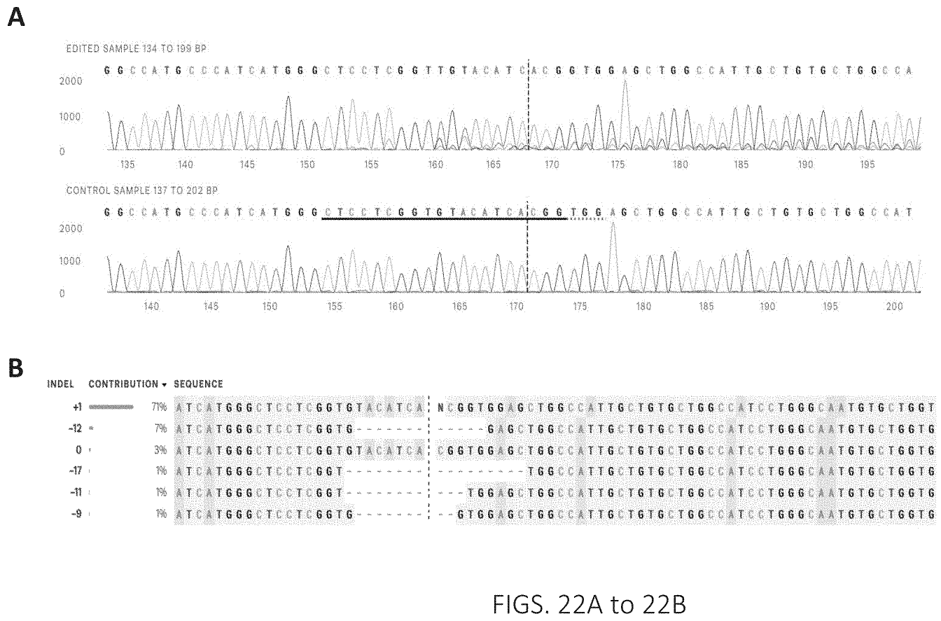

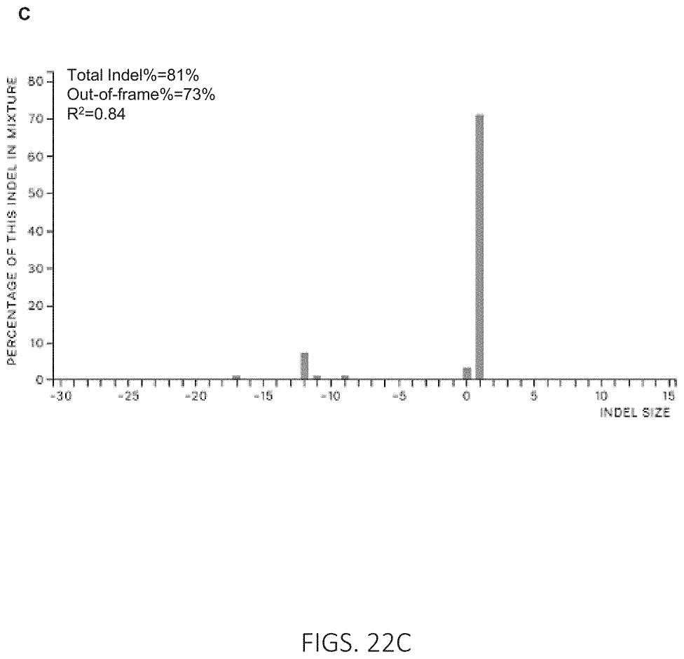

The CRISPR indel frequency and gene knock-out efficiency of the transfected iPSCs were analysed by ICE assay as described in Example 2. The ICE assay result showed that the gRNAs create indels at high frequency, including out-of-frame indels in iPSCs (FIGS. 18A-18C for RC3H1,2 KO and FIGS. 22A-22C for A2AR KO).

In summary, our data demonstrate that gene edited iPSCs describe herein can differentiate into CD34+/HE/HSC and then to immune cells such as NK, NKT or T cells using known methods for subsequent use in cancer therapy.

### Example 11—Differentiation of RC3H1 and RC3H2 KO (RC3H1,2 KO) iPSCs into iNK Cells (Edited iNK Cells)

iCD34+ Cells

The receptor CD34 is expressed on HE and HSCs, which are stem cell sources that form the platform to create immune cells. The differentiation of iPSC to CD34+ cells is a prerequisite and imperative to be able to create iPSC-derived immune cells (Sturgeon et al. Nature Biotechnology, 2014 vol 32 (6) p 554-561, Knorr et al. STEM CELLS Translational Medicine vol 2 (4) p 274-283, Zeng et al, Stem Cell Reports, 2017 vol 9 (6) p 1796-1812). Characterisation of CD34+ expression as an intermediate cell type between iPSC and immune cells is considered common practice and a key step to demonstrate the inclusion of the gene-KO in the iPSC is not disrupting any potential differentiation pathways during the initial development.

Non-transfected and transfected iPSCs (containing RC3H1,2 KO) were differentiated toward iCD34+ cells using STEMdiff™ Hematopoietic Kit (STEMCELL Technologies) following the manufacturer's instructions. Cells were isolated and stained using antibodies targeting CD34 (Miltenyi Biotec), following manufacturer recommendations. The cells were analysed via MACSQuant® flow cytometer (Miltenyi Biotec) with unstained samples and appropriate isotype controls. Dead cells (via PI staining), debris and doublets were gated out; data analysis was performed using FlowLogic™.

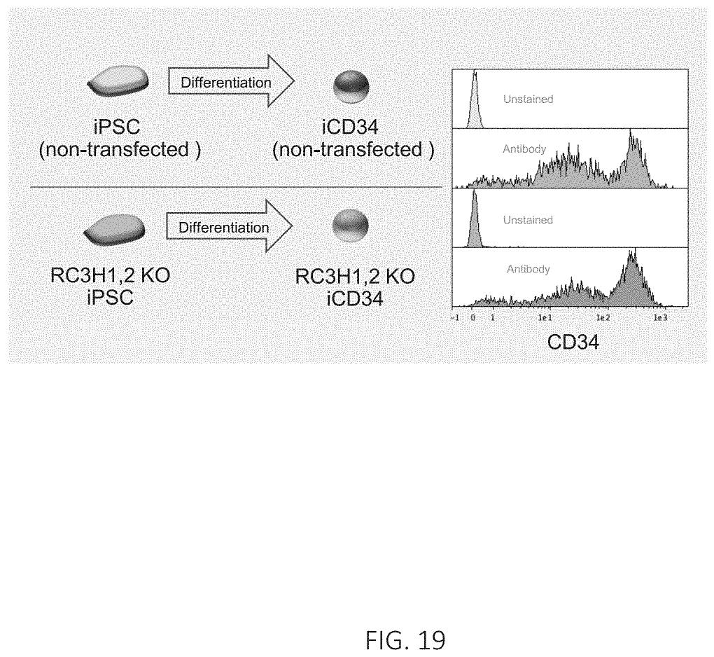

iPSCs, with or without the inclusion of RC3H1,2 KO (FIG. 19) were differentiated into iCD34+ cells. These data demonstrate successful creation of iCD34+ cells from RC3H1,2 KO iPSCs and indicate that the key development pathways required to transition from an iPSC through all the intermediate phenotypes into a population of cells containing CD34 expressing cells remain intact.

### iNK Cells

iPSCs containing RC3H1,2 KO are able to differentiate to iNK immune cells.

Gene knock-out iCD34+ (derived from RC3H1,2 KO iPSC) were further differentiated into iNK cells driven by a combination of cytokines including IL-15, FLT3, and IL-7. iNK cells can be made using published methods such as the one described in U.S. Pat. No. 9,260,696 B2 (Kaufman, Knorr), by Li et al. (Stem Cell, 23 (2018) 181-197), or using commercially available culture systems StemSpan™ NK Cell Generation Kit (Stem Cell Technologies).

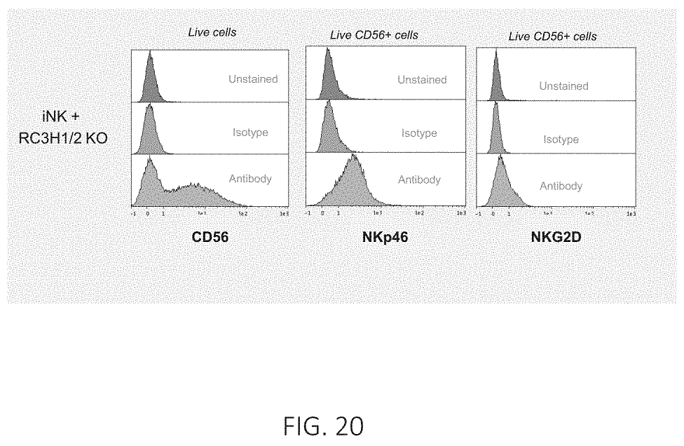

Differentiated cells were isolated and stained using antibodies targeting CD56 (Miltenyi Biotec), NKp46 (Miltenyi Biotec) and NKG2D (Miltenyi Biotec) following manufacture recommendations. The differentiated cells were analysed via MACSQuant™ flow cytometer (Miltenyi Biotec) with unstained samples and appropriate isotype controls. Dead cells (via PI staining), debris and doublets were gated out, data analysis was performed using FlowLogic™ (FIG. 20). The expression of NK functional receptors (NKp46 and NKG2D) supports that the iCD56+ cells are capable of NK-specific cytotoxic function.

### Example 12—Differentiation of A2AR KO iPSCs into iNK Cells (Edited iNK Cells)

iCD34+ Cells

Non-transfected and transfected iPSCs (containing A2AR KO) were differentiated toward iCD34+ cells using STEMdiff™ Hematopoietic Kit (STEMCELL Technologies) following the manufacturer's instructions. Cells were isolated and stained using antibodies targeting CD34 (Miltenyi Biotec), following manufacturer recommendations. The cells were analysed via MACSQuant® flow cytometer (Miltenyi Biotec) with unstained samples and appropriate isotype controls. Dead cells (via PI staining), debris and doublets were gated out; data analysis was performed using FlowLogic™.

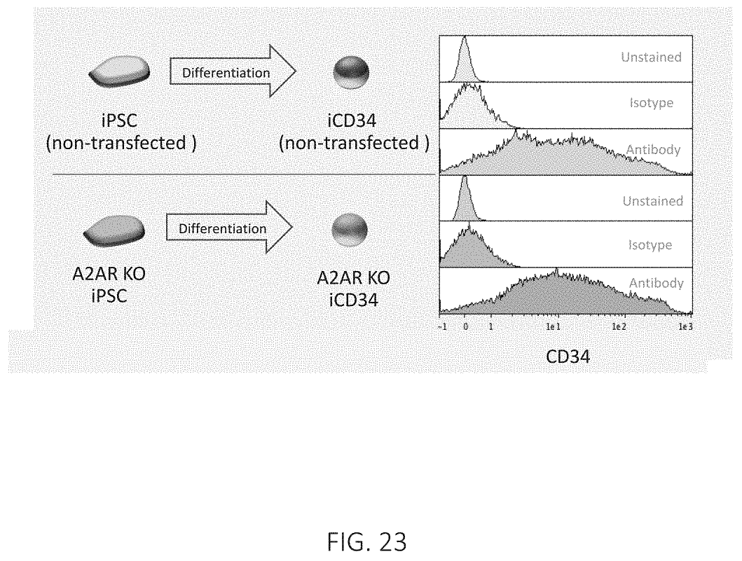

iPSCs, with or without the inclusion of A2AR KO (FIG. 23), were successfully differentiated into iCD34+ cells. These data demonstrate creation of iCD34+ cells from A2AR KO iPSCs, and indicate that the key development pathways required to transition from an iPSC through all the intermediate phenotypes into a population of cells containing CD34 expressing cells remain intact.

### iNK Cells

iPSCs containing A2AR KO are able to differentiate to iNK immune cells.

Non-transfected iCD34 (derived from non-transfected iPSC) and gene knock-out iCD34+ (derived from gene knock-out iPSC) were further differentiated into iNK cells driven by a combination of cytokines including IL-15, FLT3, and IL-7. iNK cells can be made using published methods such as the one described in U.S. Pat. No. 9,260,696 B2 (Kaufman, Knorr), by Li et al. (Stem Cell, 23 (2018) 181-197), or using commercially available culture systems StemSpan™ NK Cell Generation Kit (Stem Cell Technologies).

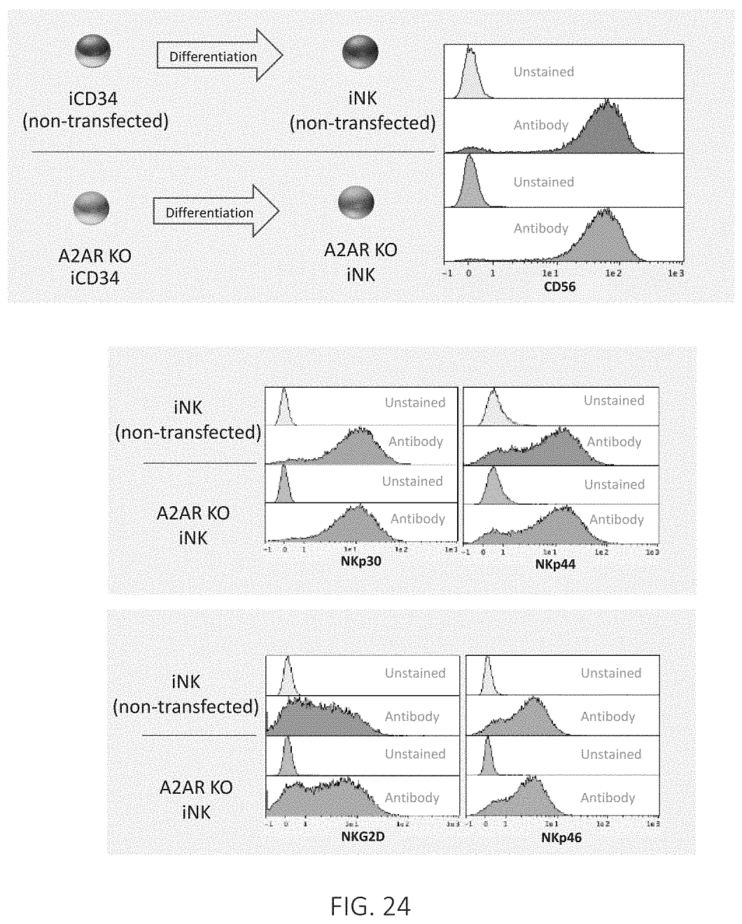

Differentiated cells were assessed for the expression of NK cell markers by flow cytometry. Dead cells, debris and doublets were gated out, such that the CD56+ histograms presented in FIG. 24 show all live cells in culture generated from either the non-transfected or transfected iPSCs samples. Unstained samples are presented to show clear positive staining of each antibody for each respective receptor. Appropriate isotype controls were negative. The expression of NK functional receptors (NKp46, NKp30, NKp44 and NKG2D) confirms that the iCD56+ cells are iNK cells capable of cytotoxic function.

### Example 13—Function of Edited iNK Cells

A2AR KO iPSCs were generated (Example 10) and differentiated to edited iNK cells (Example 12). iNK cells were then collected after 20-40 days and used in subsequent functional assays.

The ability of iNK cells to kill cancer cells was evaluated in vitro using the real-time cell monitoring system (xCELLigence®). Target cells (10,000 per 100 uL) (for example the ovarian cancer cell line OVCAR-3) were resuspended in culture media (for example, RPMI-1640 with L-glutamine basal media) supplemented with 10-20% FBS and bovine insulin and dispensed into RTCA plates. Target cells were maintained at 37° C., 5% CO₂ for at least 5 hrs to allow for cellular attachment. Following attachment of target cells, iNK effector cells were added at an E:T ratio of 1:1. In parallel, iPSC derived iNK cells were co-cultured with target cells to demonstrate the background functionality of non-transfected iNK cells in vitro. All co-cultures were maintained in optimal growth conditions for at least 10 hrs. Throughout, cellular impedance was monitored and presented herein as NCI where normalisation occurs to the time of addition of effector cells. Percent cytotoxicity (% cytotoxicity) of iNK±A2AR KO effector cells (test) relative to target cells alone (control) was calculated following 5 hrs and 10 hrs co-culture using the following equation:

((Normalised Cell Index_{control}−Normalised Cell Indexₜₑₛₜ)/Normalised Cell Index_{control})×100

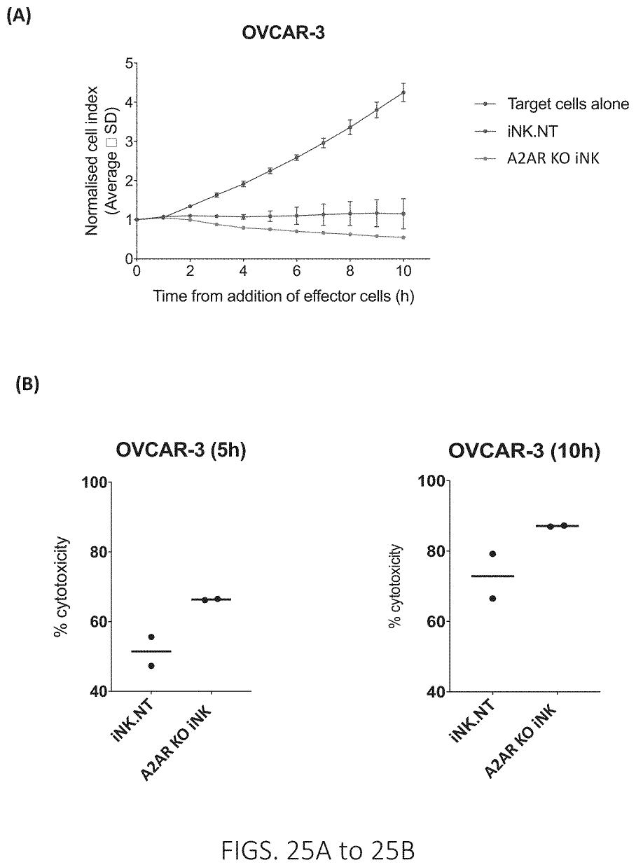

To compare the capacity of A2AR KO iPSC-derived iNK cells (A2AR KO iNK cells) to lyse tumor cells, tumor cell lines were incubated with A2AR KO iNK cells or NT iNK cells and the in vitro cytotoxicity was monitored by xCELLigence®. NT iNK cells demonstrated a cytotoxic effect when co-cultured with OVCAR-3 target cells (FIG. 25A). This effect was improved with A2AR KO iNK cells compared to the non-transfected control, demonstrating an enhancement of function in vitro. Furthermore, cytotoxicity following 5 hrs of co-culture (FIG. 25B, left side) and 10 hrs of co-culture both showed higher cytotoxicity in A2AR KO iNK (FIG. 25B, right side). Taken together, these data indicate that A2AR KO could enhance the antitumor activity not only in T cells but also in iNK cells as compared to non-transfected control cells.

Various publications, including patents, patent applications, published patent applications, accession numbers, technical articles and scholarly articles are cited throughout the specification. Each of these cited publications is incorporated by reference, in its entirety and for all purposes, in this document.

What is claimed is:

1. A method for enhancing the function of an immune cell comprising:
modifying the immune cell to inhibit the function of at least one gene selected from the group consisting of RC3H1, RC3H2, A2AR, FAS, TGFBR1 and TGFBR2.

2. A method of modifying a stem cell capable of differentiating to an immune cell comprising:
modifying the stem cell to inhibit the function of at least one gene selected from the group consisting of RC3H1, RC3H2, A2AR, FAS, TGFBR1 and TGFBR2.

3. The method of claim 2, further comprising differentiating the modified stem cell into an immune cell, wherein the function of said at least one gene is inhibited in the immune cell.

4. The method of any one of claims 1-3, wherein inhibition of the function of a gene is achieved by a gene editing system.

5. The method of claim 4, wherein the gene editing system is selected from the group consisting of CRISPR/Cas, TALEN and ZFN.

6. The method of claim 4, wherein the gene editing system is a CRISPR/Cas system which comprises a guide RNA-nuclease complex.

7. The method of claim 6, wherein the guide RNA targets a nucleotide sequence selected from the group consisting of: SEQ ID NO: 2 to SEQ ID NO: 16.

8. The method of claim 6, wherein the CRISPR/Cas system utilizes a guide RNA dependent nuclease selected from the group consisting of Cpf1, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

9. The method of any of the preceding claims, wherein the immune cell is selected from a T cell, an NK cell, an NKT cell, or a macrophage.

10. The method of claim 1 or 2, wherein inhibition of the function of a gene is achieved by reducing the level or function of mRNA, optionally through a small interfering RNA (siRNA), a short hairpin RNA (shRNA), a microRNA (miRNA), or an anti-sense nucleic acid.

11. The method of claim 1 or 2, wherein inhibition of the function of a gene is achieved by reducing the level or activity of the protein encoded by the gene, optionally through the use of an antibody or a small molecule.

12. The method of any of the preceding claims, wherein the modified cell produced by the method further comprises a nucleic acid encoding a chimeric antigen receptor (CAR).

13. The method of any of the preceding claims, wherein the modified immune cell produced by the method recognizes one or more target antigens.

14. The method of claim 13, wherein the target antigens are selected from the group consisting of TAG-72, CD19, CD20, CD24, CD30, CD47, folate receptor alpha (FRα), and BCMA.

15. The method of any one of claims 1-14, wherein said at least one gene is RC3H1.

16. The method of any one of claims 1-14, wherein said at least one gene is RC3H2.

17. The method of any one of claims 1-14, wherein said at least one gene is A2AR.

18. The method of any one of claims 1-14, wherein said at least one gene is FAS.

19. The method of any one of claims 1-14, wherein said at least one gene is TGFBR1.

20. The method of any one of claims 1-14, wherein said at least one gene is TGFBR2.

21. An immune cell produced by a method according to any one of claim 1 or 3-20, or differentiated from a modified stem cell produced by a method according to any one of claim 2 or 4-20.

22. A modified immune cell, wherein the function of at least one gene is inhibited in the modified immune cell, and wherein said at least one gene is selected from the group consisting of RC3H, RC3H2, A2AR, FAS, TGFBR1 and TGFBR2.

23. The modified immune cell of claim 22, wherein the inhibition of the function of a gene results from a reduction in the level or function of the mRNA transcribed from the gene, or the level or activity of the protein encoded by the gene.

24. The modified immune cell of claim 22, wherein the inhibition of the function of a gene results from a modification in the nucleic acid sequence of the gene.

25. The modified immune cell of any one of claims 22-24, wherein the modified immune cell is selected from a T cell, an NK cell, an NKT cell or a macrophage.

26. The modified immune cell of any one of claims 22-25, wherein the modified immune cell expresses a chimeric antigen receptor (CAR).

27. The modified immune cell of any one of claims 22-26, wherein the modified immune cell recognizes one or more target antigens.

28. The modified immune cell of claim 27, wherein the target antigen is selected from the group consisting of TAG-72, CD19, CD20, CD24, CD30, CD47, folate receptor alpha (FRα) and BCMA.

29. The modified immune cell of any one of claims 22-28, wherein said at least one gene is RC3H1.

30. The modified immune cell of any one of claims 22-28, wherein said at least one gene is RC3H2.

31. The modified immune cell of any one of claims 22-28, wherein said at least one gene is A2AR.

32. The modified immune cell of any one of claims 22-28, wherein said at least one gene is FAS.

33. The modified immune cell of any one of claims 22-28, wherein said at least one gene is TGFBR1.

34. The modified immune cell of any one of claims 22-28, wherein said at least one gene is TGFBR2.

35. A modified stem cell, capable of differentiating to an immune cell, comprising a modification in the nucleic acid sequence of at least one gene, wherein the modification inhibits the function of said at least one gene and wherein said at least one gene is selected from the group consisting of RC3H1, RC3H2, A2AR, FAS, TGFBR1 and TGFBR2.

36. The modified stem cell of claim 35, being an induced pluripotent stem cell.

37. The modified stem cell of claim 36, wherein the induced pluripotent stem cell is generated from a donor cell homozygous for three HLA genotypes.

38. The modified stem cell of any one of claims 35-37, further comprising a nucleic acid encoding a chimeric antigen receptor (CAR).

39. The modified stem cell of any one of claims 35-38, wherein said at least one gene is RC3H1.

40. The modified stem cell of any one of claims 35-38, wherein said at least one gene is RC3H2.

41. The modified stem cell of any one of claims 35-38, wherein said at least one gene is A2AR.

42. The modified stem cell of any one of claims 35-38, wherein said at least one gene is FAS.

43. The modified stem cell of any one of claims 35-38, wherein said at least one gene is TGFBR1.

44. The modified stem cell of any one of claims 35-38, wherein said at least one gene is TGFBR2.

45. A composition for enhancing the function of an immune cell, comprising: a guide RNA-nuclease complex capable of editing the sequence of a target gene, wherein the guide RNA targets a nucleotide sequence selected from the group consisting of SEQ ID NO: 2 to SEQ ID NO: 16.

46. The composition of claim 45, wherein the nuclease comprises at least one protein selected from the group consisting of Cpf1, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

47. A method of treating a condition in a subject, comprising administering to the subject a modified immune cell according to any one of claims 21-34.

48. The method of claim 47, wherein the condition is a cancer, an infection, an autoimmune disorder, fibrosis of an organ, or endometriosis.
